(19) United States
(12) Patent Application Publication
(10) Pub. No.: US 2025/0120616 A1
(43) Pub. Date: Apr. 17, 2025

(54) LASER-INDUCED GRAPHENE NON-ENZYMATIC GLUCOSE SENSORS FOR ON BODY MEASUREMENTS

(71) Applicant: The Penn State Research Foundation, University Park, PA (US)

(72) Inventor: Huanyu Cheng, State College, PA (US)

(21) Appl. No.: 18/686,479

(22) PCT Filed: Aug. 24, 2022
(86) PCT No.: PCT/US2022/075397

§ 371 Date: Feb. 26, 2024

Related U.S. Application Data
(60) Provisional application No. 63/260,813, filed on Sep. 1, 2021.

(51) Int. Cl. A61B 5/145

(52) U.S. Cl. CPC A61B 5/14532; A61B 5/14517

(57) ABSTRACT

Embodiments relate to a non-enzymatic glucose sensor. The non-enzymatic glucose sensor comprises one or more electrodes, a microfluidic channel, and at least one inlet, wherein the at least one inlet is configured to deliver a fluid to a microfluidic channel and wherein the microfluidic channel is configured to transport the fluid to the one or more electrodes. At least one of the one or more electrodes is a laser-induced graphene electrode, wherein the laser-induced graphene electrode comprises one or more uniform coatings of metal.

### CROSS-REFERENCE TO RELATED APPLICATIONS

This application claims priority to U.S. Provisional Patent Application No. 63/260,813, which was filed on Sep. 1, 2021. The entirety of this application is incorporated by reference herein.

### STATEMENT REGARDING FEDERALLY SPONSORED RESEARCH DEVELOPMENT

This invention was made with government support under Grant No. ECCS1933072 awarded by the National Science Foundation and under Grant No. HL154215 awarded by the National Institutes of Health. The Government has certain rights in the invention.

### FIELD OF THE INVENTION

Embodiments relate to a glucose sensor, particularly to a laser-induced graphene non-enzymatic glucose sensor for on body measurements.

### BACKGROUND OF THE INVENTION

Diabetes poses a major health concern, and its monitoring and therapy have been of high interest. However, the functional recovery of insulin secretion in diabetes patients is challenging, so it is crucial to continuously monitor a patient's blood glucose concentration for timely treatment with the injection of artificially synthesized insulin. Although noninvasive optical and spectroscopic measurements of glucose have been explored, these methods are associated with expensive equipment setup and are subject to interferences from movement and temperature. Due to its simplicity, electrochemical analysis has been extensively employed in portable glucose sensors for daily monitoring and clinical diagnostics. In the electrochemical setup, glucose is oxidized by an oxidizer at the working electrode to give away electrons, leading to current flow with a magnitude directly proportional to the glucose concentration. After obtaining the calibration curve in the glucose concentration versus current density, the electrochemical glucose sensor can be readily applied for use.

Both enzymatic and non-enzymatic glucose sensors have been widely used for glucose measurements. Glucose oxidase (GOx), as a representative enzymatic oxidizer, degrades rapidly even in mild conditions. In contrast, the non-enzymatic glucose sensors based on metals or metal oxides showcase excellent stability, extended operating duration, and extraordinarily harsh environment tolerance.

Although the sensitivity of non-enzymatic glucose sensors is already much higher than their enzymatic counterparts, it is still highly desirable to further improve the sensitivity for the detection of trace glucose in sweat and other biofluids. As an alternative to materials innovations, nanomaterials or 3D structures with a high specific surface areas have been shown to improve the sensitivity of non-enzymatic glucose sensors over their planar counterparts. However, the methods currently used to create 3D structures are complex and not suitable for wearable applications (e.g., due to rigid electrodes). Further, alternative methods using sensing materials with binders reduces the catalytic activity of sensing materials and compromise overall performance.

Laser-induced graphene (LIG) displays porous structures, decent conductivity, and electrochemical stability and appears to provide an excellent platform for electrochemical sensing applications. Although LIG electrodes with electroplated metals have been leveraged for non-enzymatic glucose sensors, electroplating is only suitable for large LIG electrodes due to the limited conductivity. The limited and anisotropic conductivity in the porous 3D structure also leads to a long electroplating time and non-uniform electroplated layers. In addition, clamping to electrodes during electroplating may easily lead to the falling-off of LIG, leading to a failure of electroplating. Efforts have been devoted to increasing the conductivity and mechanical robustness of LIG (e.g., spray coating a layer of conductive polymer as a binder). However, the binder layer negatively affects the porous structure to result in low coverage of sensing metals for reduced sensitivity. Further, previous work on wearable non-enzymatic glucose sensors is based on the reduction of protons to achieve a basic environment, which may cause a hazard to patients.

Conventional methods and apparatuses can be appreciated from Gao et al., “Electrochemical Detection of Glucose Molecules Using Laser-Induced Graphene Sensors: A Review” (Apr. 16, 2021); Huang et al., “Laser-Induced Graphene: En Route to Smart Sensing” (Aug. 3, 2020); Vivaldi et al., “Three-Dimensional (3D) Laser-Induced Graphene: Structure, Properties, and Application to Chemical Sensing” (Jun. 24, 2021); Zhang et al., “A flexible non-enzymatic glucose sensor based on copper nanoparticles anchored on laser-induced graphene” (Oct. 2, 2019); Simsek et al., “Carbon nanomaterial hubrids via laser writing for high-performance non-enzymatic electrochemical sensors: a critical review” (May 12, 2021); Settu et al., “Laser-Induced Graphene-Based Enzymatic Biosensor for Glucose Detection” (Aug. 20, 2021); Chinese Patent No. 109421402B to Hu et al.; and U.S. Pat. No. 9,869,653 to Chambers et al.

Accordingly, there is a need for a wearable non-enzymatic glucose sensor that utilizes a uniform metal coating process on a porous LIG electrode with electroless plating and that displays high sensitivity and decreased reliability on a basic environment.

### SUMMARY OF THE INVENTION

Embodiments relate to a non-enzymatic glucose sensor. The non-enzymatic glucose sensor comprises one or more electrodes, a microfluidic channel, and at least one inlet, wherein the at least one inlet is configured to deliver a fluid to the microfluidic channel and wherein the microfluidic channel is configured to transport the fluid to the one or more electrodes. At least one of the one or more electrodes is a laser-induced graphene electrode, wherein the laser-induced graphene electrode comprises one or more uniform coatings of metal.

In an exemplary embodiment, a non-enzymatic glucose sensor comprises one or more electrodes, a microfluidic channel, and at least one inlet. The at least one inlet is configured to deliver a fluid to the microfluidic channel, and the microfluidic channel is configured to transport the fluid to the one or more electrodes. At least one of the one or more electrodes is a laser-induced graphene electrode, wherein the laser-induced graphene electrode comprises a uniform coating of a metal.

In some embodiments, the laser-induced graphene electrode further comprises a second uniform coating of a second metal, wherein the second uniform coating is coated on the uniform coating of the metal.

In some embodiments, the metal comprises nickel the second metal comprises gold.

In some embodiments, the non-enzymatic glucose sensor further comprises a reaction member comprising a reaction cavity positioned within the reaction member. The reaction member is attached to the one or more electrodes such that reaction cavity is encapsulated.

In some embodiments, the reaction member further comprises an electrolyte solution positioned within the reaction cavity.

In some embodiments, the electrolyte solution has a pH between 8 and 12.5.

In some embodiments, the electrolyte solution comprises NaOH.

In some embodiments, the reaction member further comprises a porous polymer layer positioned within the reaction cavity, and wherein the electrolyte solution is positioned within the porous polymer layer.

In an exemplary embodiment, a non-enzymatic glucose sensor comprises a top layer, a bottom layer, and an intermediate layer. The intermediate layer comprises one or more electrodes, a microfluidic channel, and at least one intermediate inlet. The at least one intermediate inlet is configured to deliver a fluid to the microfluidic channel and the microfluidic channel is configured to transport the fluid to the one or more electrodes. At least one of the one or more electrodes is a laser-induced graphene electrode, wherein the laser-induced graphene electrode comprises a uniform coating of a metal. The intermediate layer is positioned between the top layer and the bottom layer and the bottom layer is configured to be in contact with a user.

In some embodiments, the laser-induced graphene electrode further comprises a second uniform coating of a second metal coated on the uniform coating of the metal.

In some embodiments, the metal comprises nickel the second metal comprises gold.

In some embodiments, the bottom layer comprises at least one bottom inlet configured to deliver a fluid to the at least one intermediate inlet.

In some embodiments, the bottom layer comprises an adhesive configured to attach to the user.

In some embodiments, the top layer is sealed to the bottom layer.

In an exemplary method of forming a non-enzymatic glucose sensor, the method comprises providing a substrate, providing a laser device, laser-scribing the substrate using the laser device to form one or more laser-induced graphene electrodes, depositing a uniform coating of a metal on at least one of the one or more laser-induced graphene electrodes, and providing a microfluidic channel, wherein the microfluidic channel is configured to transport a fluid to the one or more electrodes.

In some methods of forming a non-enzymatic glucose sensor, the method comprising depositing a second uniform coating of a second metal on the uniform coating of the metal.

In some methods of forming a non-enzymatic glucose sensor, the metal comprises nickel and the second metal comprises gold.

In some methods of forming a non-enzymatic glucose sensor, the method further comprises providing a reaction member comprising a reaction cavity positioned within the reaction member, attaching the reaction member to the one or more electrodes such that reaction cavity is encapsulated, providing an electrolyte solution positioned within the reaction cavity, and providing a porous polymer layer positioned within the reaction cavity, wherein the electrolyte solution is positioned within the porous polymer layer.

In some methods of forming a non-enzymatic glucose sensor the step of depositing a uniform coating of a metal on at least one of the one or more laser-induced graphene electrodes comprises electroless-plating of the metal on at least one of the one or more laser-induced graphene electrodes.

In some methods of forming a non-enzymatic glucose sensor, the step of depositing a second uniform coating of a second metal on the coating of the metal comprises electroless-plating of the second metal on the uniform coating of the metal.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 shows an exemplary non-enzymatic glucose sensor with an encapsulated reaction cavity.

FIG. 2 shows an exemplary fabrication process of a non-enzymatic glucose sensor, namely laser scribing a flexible substrate to prepare LIG electrodes.

FIG. 3 shows an exemplary reaction member comprising a reaction cavity and a porous polymer layer.

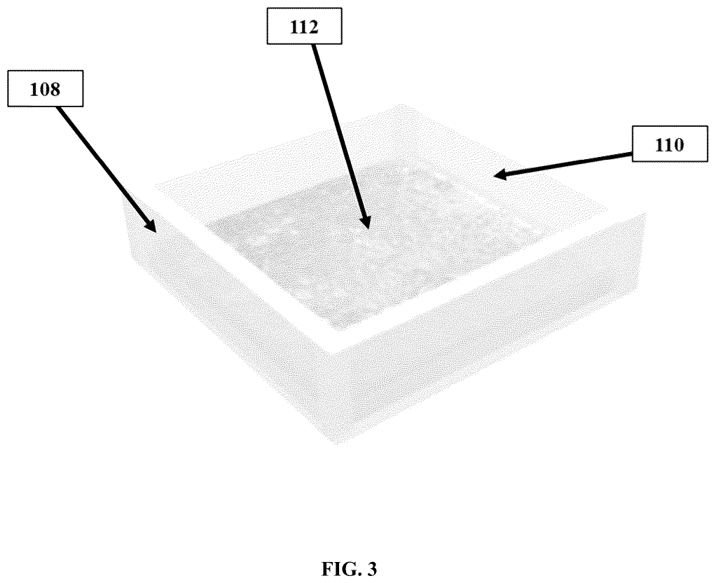

FIG. 4 shows a cross-section view of an exemplary electrode uniformly coated with one or more metals.

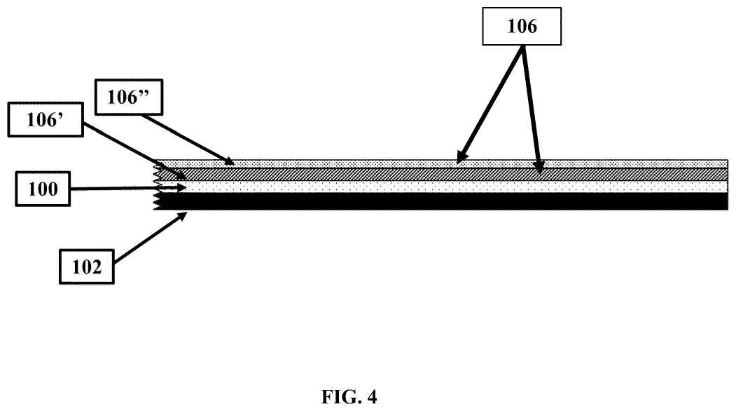

FIG. 5 shows a microfluidic channel for sweat sampling in a non-enzymatic glucose sensor and marks in the microfluidic channel for the calculation of flow rate.

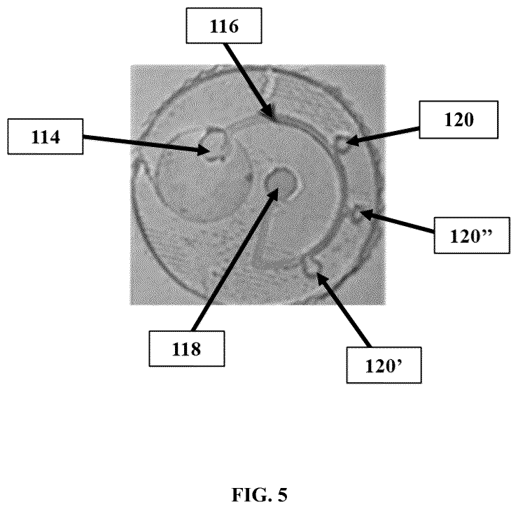

FIG. 6 shows an exemplary embodiment of a non-enzymatic glucose sensor comprising a top layer, an intermediate layer, and a bottom layer.

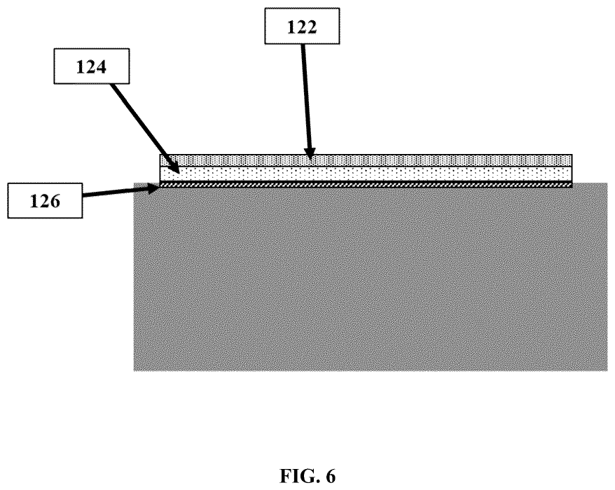

FIG. 7 shows an exemplary fabrication process of a reference electrode, including (i) spray coating of silver nanoparticle (AgNP) ink on LIG, (ii) photonic sintering of AgNP ink by xenon light pulses, (iii) immersion of the Ag/LIG electrode in a FeCl₃ solution for 3 minutes, resulting in chlorination, and (iv) rinsing in deionized (DI) water, drying at room temperature, and dropping another Nafion layer onto the AgCl/Ag/LIG electrode.

FIG. 8 shows an open circuit potential of a fabricated Ag/AgCl reference electrode in 30 days' measurement.

FIG. 9 shows an exemplary fabrication process of a reaction member comprising a reaction cavity and a porous polymer layer, including (i) uniformly dispersing salt crystals at the bottom of a rectangular PDMS frame, (ii) pouring diluted Ecoflex by heptane (weigh ratio of 1:1) into the cavity to immerse the salt layer, (iii) immersing the cavity immersed in water to dissolve the salt template after Ecoflex was cured, and (iv) rinsing in DI water and drying at room temperature.

FIG. 10 shows a Raman spectrum of LIG and a polyimide flexible substrate.

FIG. 11 shows a top view of a scanning electron microscope (SEM) image of a LIG working electrode (scale bar is 10 μm).

FIG. 12 shows electroplating of Ni on LIG.

FIG. 13 shows top views of scanning electron microscope (SEM) images of Ni 1/LIG, Au 1/Ni 1/LIG, Ni 3/LIG, and Au 1/Ni 3/LIG working electrodes (scale bar is 10 μm).

FIG. 14 shows magnified scanning electron microscope (SEM) images of Ni_1, Au/Ni_1, Ni_3, and Au/Ni_3 electrodes (scale bar is 1 μm).

FIG. 15 shows XPS measurements of LIG, Ni_1/LIG, and Au_1/Ni_1/LIG working electrodes.

FIG. 16 shows a chronoamperometry response of working electrodes with different Ni plating time under an applied potential of 0.50 V in 0.05 M NaOH solutions.

FIG. 17 shows EIS measurements of working electrodes with a different Ni plating time in 0.05 M NaOH solutions.

FIG. 18 shows XRD measurements of LIG, Ni_1/LIG, and Au_1/Ni_1/LIG working electrodes.

FIG. 19 shows FTIR spectra of LIG, Ni_1/LIG and Au 1/Ni 1/LIG.

FIG. 20 shows cyclic voltammetry (CV) curves of four different working electrodes in 0.05 M NaOH solutions with a scan rate of 50 mV/s.

FIG. 21 shows CV curves of a Au_1/Ni_1/LIG working electrode in 0.05 M NaOH solutions with a scan rate of 20 or 50 mV/s (inset highlights the two oxidation peaks from Ni and Au).

FIG. 22 shows a chronoamperometry response of a Ni_1/LIG working electrode with successive addition of glucose into gently stirred 0.05 M NaOH solutions under the applied potential of 0.45, 0.50, and 0.55 V.

FIG. 23 shows magnified scanning electron microscope (SEM) images of a Ni/LIG electrode after electroless plating of Ni for 10 seconds (Ni_10 s) and 30 seconds (Ni_30 s) (scale bar is 1 μm).

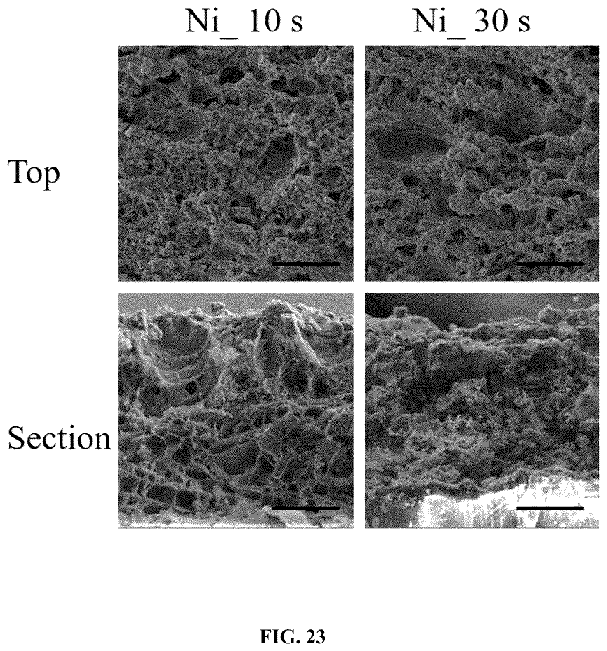

FIG. 24 shows EIS measurements of working electrodes with a different Au plating time in 0.05 M NaOH solutions.

FIG. 25 shows a chronoamperometry response of an Au_1/Ni_1/LIG working electrode with successive addition of glucose into gently stirred 0.05 M NaOH solutions under the applied potential of 0.45, 0.50, and 0.55.

FIG. 26 shows a chronoamperometry response of working electrodes with Ni plating for 10 seconds or 30 seconds under an applied potential of 0.50 V in 0.05 M NaOH solutions.

FIG. 27 shows STEM-EDS characterization of an Au 1/Ni 1/LIG working electrode.

FIG. 28 shows a chronoamperometry response of working electrodes with different Au plating time under an applied potential of 0.50 V in 0.05 M NaOH solutions.

FIG. 29 shows a chronoamperometry response of the Ni_1/LIG and Au 1/Ni 1/LIG under an applied potential of 0.1 V in 0.05 M NaOH solutions. The linear regression coefficient is 0.991 and 0.992 for the black and red line, respectively.

FIG. 30 shows optical images of the LIG foam and LIG fibers.

FIG. 31 shows current density as a function of the glucose concentration for the LIG foam-based and LIG fiber-based glucose sensors.

FIG. 32 shows current density as a function of the glucose concentration of an Au/Ni glucose sensor without a reaction cavity in the solution with different pH levels (e.g., 10.5 or 12.5).

FIG. 33 shows CV curves of an Au/Ni glucose sensor without a reaction cavity in the solution with different pH levels (e.g., 10.5 or 12.5).

FIG. 34 shows current density as a function of the glucose concentration of an Au/Ni glucose sensor with a reaction cavity in the solution with different pH levels (e.g., 10.5 or 12.5).

FIG. 35 shows a chronoamperometry response of an Au/Ni glucose sensor under deformations.

FIG. 36 shows current density versus glucose concentration of a flexible glucose sensor in a flat or bending state measured in 0.05 M NaOH solutions.

FIG. 37 shows selectivity of the flexible glucose sensor against various interfering substances.

FIG. 38 shows CV curves of an Au/Ni_1/LIG glucose sensor in 0.05 M NaOH or KOH solutions.

FIG. 39 shows a comparison of the measured glucose in cell culture media between commercial and demonstrated glucose sensors.

FIG. 40 shows CV curves of an Au/Ni_1/LIG glucose sensor in 0.05 or 0.1 M NaOH solutions.

FIG. 41 shows selectivity of the flexible glucose sensor against various interfering substances, including KCl, CaCl2, NaHCO3, lactate, and glycine.

FIG. 42 shows CV curves of an Au/Ni glucose sensor with a reaction cavity in the solution with different pH levels (e.g., 10.5 or 12.5).

FIG. 43 shows CV curves of an Au_1/Ni_1/LIG glucose sensor in a solution with a pH of 10.5 or 10.0.

FIG. 44 shows a comparison of the water evaporation rate between an open cavity and the one with a porous Ecoflex encapsulation.

FIG. 45 shows a comparison of the contact angle between solid and porous Ecoflex substrates.

FIG. 46 shows sweat glucose concentration after a meal measured by titration into the cavity, the wearable fluidic device, and commercial glucose sensor.

FIG. 47 shows an optical image of an encapsulated glucose sensor attached to the arm of a healthy human subject.

FIG. 48 shows glucose measurement of the encapsulated flexible glucose sensor.

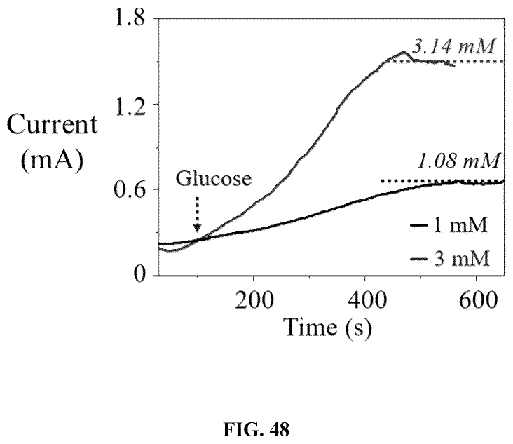

FIG. 49 shows open circuit voltage between a PANI/LIG working electrode and a Nafion/AgCl/Ag/LIG reference electrode in buffer solutions with different pH levels from three measurements.

FIG. 50 shows a chronoamperometry response of an encapsulated glucose sensor to human sweats collected 1 hour or 3 hours after a meal from a healthy subject.

FIG. 51 shows integrated devices with a microfluidic channel for sweat sampling and non-enzymatic glucose sensors, along with the hydrophilic properties of PDMS with the air plasma and PVP treatment and marks in the microfluidic channel for the calculation of flow rate.

FIG. 52 shows an optical image to show the electrical connection between the sensor electrode and the electrochemical station via copper wires and silver paste, which is further encapsulated by a thin layer of PDMS.

FIG. 53 shows hydrophilic properties of PDMS after air plasma and PVP treatment.

FIG. 54 shows an exemplary use of an embodiment of the sensor with a Bluetooth enabled device.

### DETAILED DESCRIPTION OF THE INVENTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

Embodiments relate to a non-enzymatic sensor 1000. In exemplary embodiments, the non-enzymatic sensor 1000 may comprise one or more electrodes 100. As used herein, one or more electrodes 100 generally describes and includes 100a, 100b, 100c, and/or 100d. The one or more electrodes 100 may be electrically connected to each other. The one or more electrodes 100 may comprise a working electrode 100a, a reference electrode 100b, and/or a counter electrode 100c. It is understood that a working electrode 100a may monitor the oxidation or reduction of a solution in contact with or near the surface of the electrode. It is understood that a reference electrode 100b may provide a stable potential for controlled regulation of the working electrode 100a potential and allow the measurement of the potential of the working electrode 100a without passing current through the reference electrode 100b. It is understood that a counter electrode 100c (or auxiliary electrode) may establish a connection to a solution such that a current may be applied to a working electrode 100a. The one or more electrodes 100 may be configured to perform electro oxidation of a solution and to convert an electrochemical reaction into a measureable electrical signal. For example, with respect to glucose, glucose may be oxidized by an oxidizer at the working electrode 100a to give away electrons, leading to a current flow with a magnitude directly proportional to the glucose concentration. The one or more electrodes 100 may be connected to an electrochemical workstation, such that information collected by the one or more electrodes 100 may be stored and/or analyzed.

In exemplary embodiments, any number of the one or more electrodes 100 may be a laser-induced graphene (LIG) electrode 100d. The LIG electrode 100d may be formed by laser-scribing a flexible substrate 102 using a laser 104 (e.g., CO₂ laser, Yttrium Aluminum Garnet laser, etc.). Exemplary embodiments discussed herein discuss use of a CO₂ laser, but it is understood that other lasers can be used. The CO₂ laser 104 may be used to scribe any programmable shape and/or configuration into the flexible substrate 102. It is contemplated that the LIG may be in the form of LIG foam or LIG fibers. The flexible substrate 102 may be polyimide (PI) film or any other suitable substrate (e.g., polyethylene terephthalate (PET), polyethylene naphthalate (PEN), etc.). The flexible substrate 102 may have a thickness of 25-150 μm, 50-125 μm, or 75-100 μm.

It is contemplated that the LIG comprises a three-dimensional porous structure. The three-dimensional porous structure may increase the specific surface area of the LIG electrode 100d and may improve the sensitivity of the LIG electrode 100d relative to planar sensing materials such that the LIG electrode 100d may detect trace amounts of glucose in biofluids (e.g., blood, interstitial fluid, etc.). It is contemplated that the LIG electrode 100d may detect glucose concentrations of 0.01-1.11 mM in sweat and 0.2-0.92 mM in tears.

It is contemplated that any number of the one or more electrodes 100 may comprise one or more coatings of metal 106. Any one or more of coatings can cover a portion of the electrode 100 or the entire electrode 100. As used herein, one or more coatings 106 generally describes and includes 106′ and/or 106″. Any one or more of coatings can be uniform. It is understood that uniform means substantially consistent and/or substantially homogeneous in material or chemical composition, material or chemical concentration, crystalline structure, thickness (e.g., consistent thickness on the entire electrode surface upon which the coating is deposited), etc. It is contemplated that a uniform coating of metal 106 may increase the sensitivity of an electrode 100 and may increase the linear range for glucose sensing relative to other embodiments. A uniform coating of metal 106 may be deposited on an electrode 100 such that the metal may be an outermost layer. For example, if the electrode 100 is an LIG electrode 100d, the metal may be deposited on the LIG. It is contemplated that a coating 106 may be deposited on an electrode 100 using electroless plating, electroplating, etc. For example, one may use electroless plating metal precursors to plate an electrode 100. One or more uniform coatings 106 may comprise the same metals or different metals. For example, a first coating may comprise a first metal, and a second coating may comprise a second metal. The metal of first coating may be the same as the metal of the second coating or the second metal may be a different metal than the metal of the first coating. The metal may be nickel, gold, copper, platinum, cobalt, iron, titanium, or any suitable metals or metal oxides or mixtures thereof.

In a preferred embodiment, an electrode 100 (e.g., a LIG electrode 100d) may comprise a uniform coating of a first metal 106a and a uniform coating of a second metal 106b. In a more preferred embodiment, the first metal 106a is nickel and the second metal 106b is gold. It is contemplated that a uniform coating of gold may mitigate the potential allergic reaction (e.g., an allergic reaction to other metals present, such as nickel) in certain patient populations and may further enable a large linear sensing range (e.g., 0-30 mM under a small bias voltage (e.g., 0.1 V). In a preferred embodiment, the uniform coating of the first metal 106a (e.g., nickel) may first be deposited on an electrode 100 and the uniform coating of the second metal 106b (e.g., gold) may be deposited on the uniform coating of the first metal 106a (e.g., nickel), such that the uniform coating of the second metal 106b (e.g., gold) may be an outermost layer. While it is contemplated that the uniform coating of the first metal 106a may be thicker than the uniform coating of the second metal 106b, the uniform coating of the first metal 106a may be thinner than the uniform coating of the second metal 106b, the uniform coating of a portion of the first metal 106a may be thinner than a portion of the second metal 106b while another portion of the first metal 106a may be thicker than a portion of second metal 106b, the uniform coatings of each may have the same thickness, etc.

In exemplary embodiments, the reference electrode 100b may be a LIG electrode 100d. In a preferred embodiment, the reference electrode 100b may comprise one or more uniform coatings of silver, chlorine, or mixtures thereof.

In exemplary embodiments, the counter electrode 100c may be a LIG electrode 100d. In a preferred embodiment, the counter electrode 100c may comprise no uniform coatings and/or no further modification.

It is contemplated that the one or more electrodes 100 may be positioned (e.g., formed in, deposited on, attached to, etc.) on the same substrate 102 (e.g., on the same film).

It is contemplated that the one or more electrodes 100 may be flexible, such that the one or more electrodes 100 may be used in a wearable application. For example, the one or more electrodes 100 may accommodate any body part and/or any contour and function while deformed and/or stressed.

In exemplary embodiments, the non-enzymatic sensor 1000 may further comprise a reaction member 108. The reaction member 108 may be formed from a flexible polymer, such as polydimethylsiloxane (PDMS) or any other suitable polymer. The reaction member 108 may comprise a reaction cavity 110, wherein the reaction cavity 110 is a hollowed-out portion (e.g., an empty space enclosed within the reaction member 108, but open on a surface of the reaction member 108) of the reaction member 108. The reaction member 108 may be of any shape, but it is contemplated that the reaction member 108 may be a three-dimensional shape such that the reaction member 108 may support a reaction cavity 110.

The reaction member 108 may comprise a porous polymer layer 112. It is contemplated that the porous polymer layer 112 may be positioned within the reaction cavity 110. The porous polymer layer 112 may be formed from any polymer or mixtures of polymers, such as silicone polymers or any other suitable polymer or mixtures of polymers.

The reaction member 108 may further comprise an electrolyte solution. It is contemplated that the electrolyte solution may be positioned within the reaction cavity 110 of the reaction member 108. It is further contemplated that the electrolyte solution may be positioned within the porous polymer layer 112 of the reaction member 108. For example, the electrolyte solution may be soaked into the porous polymer layer 112, such that the electrolyte solution remains within the porous polymer layer 112 no matter the orientation (e.g., upside down) or bending of the reaction member 108. It is understood that the electrolyte solution may facilitate the electrochemical reaction (e.g., provide a basic environment for sensing). The electrolyte solution may be an alkali solution, such as NaOH, KOH, or any other suitable solution. In a preferred embodiment, the electrolyte solution may have a pH between 8.0 and 12.5, and more preferably 10.5 and 12.5. It is contemplated that the electrolyte solution's position within the reaction cavity 110 may provide a stable pH environment and reduce evaporation of the electrolyte solution, leading to longer durability and advantageous characteristics for wearable applications. The reduced requirement for a highly basic environment (e.g., pH≥13-14 as compared to the pH requirements of the disclosed subject matter) further leads to an advantageous wearable application and minimizes the risk of harm for users.

It is contemplated that the porous polymer layer 112 may be perforated with a plurality of holes. It is contemplated that plurality of holes may reduce the response time of the one or more electrodes 100.

In exemplary embodiments, spent electrolyte solution may be replaced with fresh (e.g., not yet used) electrolyte solution after use (e.g., the electrolyte solution is replaceable). For example, the reaction member 108 may be cleaned with deionized (DI) water after use such that the non-enzymatic sensor 1000 may be reused for another measurement.

It is contemplated that the one or more electrodes 100 may be attached (e.g., coupled or removably coupled) to the reaction member 108, such that the one or more electrodes 100 and the reaction member 108 may form an encapsulated reaction cavity 110. It is understood that encapsulated means enclosed (e.g., surrounded on all sides). For example, the one or more electrodes 100 may act as a closed face over the otherwise open face of the reaction member 108, such that the porous polymer layer 112 of the reaction member 108 and the one or more electrodes 100 are positioned within the encapsulated reaction cavity 110. The one or more electrodes 100 may be attached to the reaction member 108 using an adhesive, such as a PDMS adhesive, a glue, or any other suitable adhesives.

It is further contemplated that the reaction member 108 may further comprise at least one inlet 114, wherein the at least one inlet 114 is configured to deliver a fluid to the reaction member 108 via a microfluidic channel 116.

In exemplary embodiments, the microfluidic channel 116 may be any shape or configuration. It is contemplated that the microfluidic channel 116 may be configured to transport a liquid to the reaction member 108. It is further contemplated that the microfluidic channel 116 may be configured to transport a liquid to the at least one inlet 114 of the reaction member 108. The microfluidic channel 116 may comprise at least one inlet 118, wherein the at least one inlet 118 is configured to deliver fluid into the microfluidic channel 116. For example, in a wearable application, the at least one inlet 118 may deliver sweat and/or other biofluids into the microfluidic channel 116. The microfluidic channel 116 may further comprise a plurality of marks 120, wherein the plurality of marks 120 are pockets of empty space positioned throughout the microfluidic channel 116. As used herein, a plurality of marks 120 generally describes and includes 120′ and/or 120″. It is contemplated that the plurality of marks 120 may be positioned in an equidistant manner from one another. As fluid enters the microfluidic channel 116, the microfluidic channel 116 and the marks 120 may fill with biofluids. The plurality of marks 120 may be configured to allow for calculating the flow rate of fluid entering the microfluidic channel 116. For example, the flow rate of the liquid may be calculated as the ratio of the microfluidic channel 116 volume between two marks (e.g., 120′ and 120″) to the corresponding filling time of the two marks (e.g., 120′ and 120″). The plurality of marks 120 may be configured to allow for calculating the liquid volume injected into the microfluidic channel 116. For example, the liquid volume may be calculated as the product of the flow rate of the liquid and the filling time of two marks (e.g., 120′ and 120″).

The microfluidic channel 116 may be formed within a polymer, such as PDMS or any other suitable polymer. It is contemplated that the polymer may be hydrophilic and/or treated to comprise hydrophilic properties.

In exemplary embodiments, the microfluidic channel 116 may be cleaned with deionized (DI) water after use such that the device may be reused for another measurement.

In exemplary embodiments, the non-enzymatic sensor 1000 may be organized into layers. Specifically, the sensor may comprise a top layer 122, an intermediate layer 124, and a bottom layer 126.

In exemplary embodiments, the top layer 122 (e.g., the capping layer) may be positioned on top of the intermediate layer 124. The top layer 122 may be attached to the intermediate layer 124 using an adhesive, such as a PDMS adhesive, a glue, or any other suitable adhesive.

In exemplary embodiments, the bottom layer 126 (e.g., the adhesive layer) may be positioned below the intermediate layer 124. The bottom layer 126 may be attached directly to a surface of interest (e.g., skin or any other human tissue), such that the bottom layer 126 may achieve intimate contact with the surface. For example, in wearable applications, the bottom layer 126 may be applied directly on skin and achieve intimate contact with the skin for efficient biofluid sampling. The bottom layer 126 may comprise at least one bottom inlet, wherein the at least one bottom inlet is configured to deliver fluid into the non-enzymatic sensor 1000.

In exemplary embodiments, the intermediate layer 124 may be positioned in between the top layer 122 and the bottom layer 126. The intermediate layer 124 may comprise the reaction member 108 as described above, the one or more electrodes 100 as described above, the microfluidic channel 116 as described above. It is contemplated that the at least one bottom inlet may be aligned with or otherwise connected to the at least one inlet 118 of the microfluidic channel 116 (e.g., an intermediate inlet) such that the fluid may be first introduced to at least one bottom inlet and consequently transported to the reaction member 108.

The top layer 122 be sealed such that the top layer 122 and the bottom layer 126 form an encapsulated non-enzymatic sensor 1000. It is contemplated that the top layer 122 may be sealed (e.g., hermetically sealed) to the intermediate layer 124 or the bottom layer 126. In a preferred embodiment, the top layer 122 is sealed after electrolyte is soaked into the porous polymer layer 112 of the reaction cavity 110.

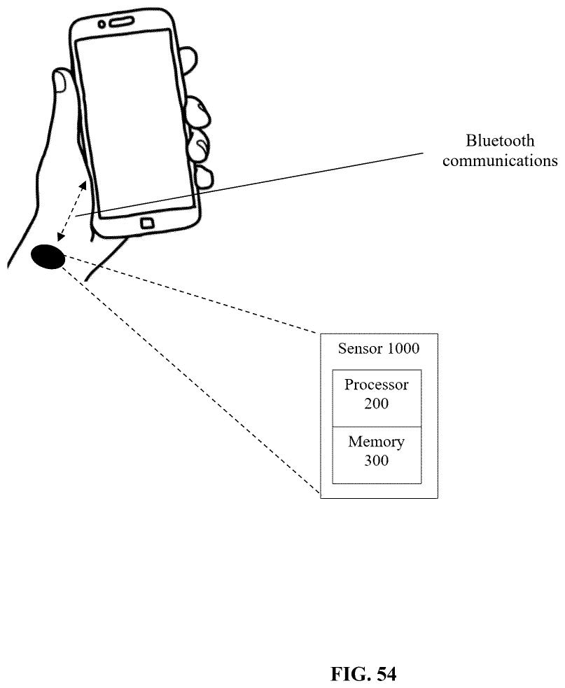

In exemplary embodiments, the non-enzymatic sensor 1000 may be configured for use with a Bluetooth device (e.g., smartphone, app, watch (e.g., smart watch), computer, etc.), such that information collected by the non-enzymatic sensor may be stored, analyzed, and/or displayed. (See FIG. 54). For instance, embodiments of the non-enzymatic sensor 1000 can be part of or in communication with a processor 200. The processor 200 can be part of or in communication with a machine (e.g., a computer device, a logic device, a circuit, an operating module (hardware, software, and/or firmware), etc.). The processor 200 can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. The processor 200 can receive, process, and/or store readings from the sensor 1000.

It should be noted that use of processors 200 herein can include any one or combination of a Graphics Processing Unit (GPU), a Field Programmable Gate Array (FPGA), a Central Processing Unit (CPU), etc. The processor 200 can include one or more processing or operating modules. A processing or operating module can be a software or firmware operating module configured to implement any of the functions disclosed herein. The processing or operating module can be embodied as software and stored in memory, the memory being operatively associated with the processor 200. A processing module can be embodied as a web application, a desktop application, a console application, etc.

The processor 200 can include or be associated with a computer or machine readable medium. The computer or machine readable medium can include memory 300. Any of the memory 300 discussed herein can be computer readable memory configured to store data. The memory 300 can include a volatile or non-volatile, transitory or non-transitory memory, and be embodied as an in-memory, an active memory, a cloud memory, etc. Embodiments of the memory 300 can include a processor module and other circuitry to allow for the transfer of data to and from the memory 300, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The computer or machine readable medium can be configured to store one or more instructions thereon. The instructions can be in the form of algorithms, program logic, etc. that cause the processor 200 to execute any of the functions disclosed herein.

The processor 200 can be in communication with other processors of other devices (e.g., a computer device, a computer system, a laptop computer, a desktop computer, etc.). An exemplary other device can be a Bluetooth enabled device, near field communication device, etc. Any of those other devices can include any of the exemplary processors disclosed herein. Any of the processors can have transceivers or other communication devices/circuitry to facilitate transmission and reception of wireless signals. Any of the processors can include an Application Programming Interface (API) as a software intermediary that allows two or more applications to talk to each other. Use of an API can allow software of the processor 200 of the system 100 to communicate with software of the processor of the other device(s). The processor 200 of the sensor 1000 can be in communication with the processor 200 of the other device. The other device can include software such as glucose monitoring software, adaptive insulin injection therapy software, etc. that uses sensor readings from the sensor 1000 as inputs. For instance, the processor 200 of the sensor 1000 can transmit signals to the processor of the other device. This can be a push or pull operation. The sensor 2000 can transmit the sensor readings continuously, periodically, as requested by a user of the other device, as-needed via determination of an algorithm embedded in the other device, etc.

### Examples

### Materials

Polyimide (PI) thin films (e.g., flexible substrates) with a thickness of 50 μm (Kapton® HN Film) were purchased from CS Hyde company. Nickel (RTM Kit) and gold (Immersion Gold CF) electroless plating precursors were purchased from Transene company. Silicone polymers, including Ecoflex 00-50 and Sylagrd 184, were purchased from Smooth-On and Dow Corning. Heptane to dilute Ecoflex, polyvinylpyrrolidone (PVP) for surface treatment, and polyaniline (PANI) for the pH measurement was purchased from Sigma-Aldrich. All chemicals were used without further purification. The cell culture media, including DMEM (11965092) and RPMI (11875119), were purchased from Thermo Fisher Scientific. Another customized cell culture medium composed of salts, amino acids, glucose, and cell-secreted factors, L7, was used to culture human pluripotent stem cells for 24 hours before the measurement.

### Fabrication of Flexible LIG Electrodes

PI film (thickness of 50 μm) was first rinsed with ethanol and DI water. After drying in the air, it was attached to an Ecoflex-coated silicon wafer for laser scribing. The flexible conductive LIG electrodes with programmable shapes in the form of foams or fibers were created by a CO₂ laser (VLS2.30, Universal Laser Systems, Inc. VLS2.3) with dual-modes (i.e., raster and vector modes). The LIG foams on PI were obtained using the raster mode with optimized settings: 10.5% of the maximum power, 11% of the maximum speed, 1000 PPI, and an image density of 6. The LIG fibers (LIGF) on PI were prepared by the same laser system with a different set of setting parameters: 50% of the maximum power, 5% of the maximum speed, 100 PPI, and an image density of 3. After a vector cutting cycle (4% of the maximum power, 7% of the maximum speed, and 1000 PPI) to cut along the pattern outline, removal of the excessive PI outside the pattern completes the fabrication of LIG foam or fiber electrodes.

### Fabrication of Counter, Reference, and Working Electrodes

The LIG electrodes were used in the sensor for all three electrodes, namely, the counter electrode (CE), reference electrode (RE), and working electrode (WE). The CE directly used the LIG electrode without further modifications. To integrate Ag/AgCl for the RE, the LIG electrode was spray-coated with a commercial silver nanoparticle (AgNP) ink (JS-A191, Novacentrix). Photonic sintering of the AgNP ink with xenon light pulses evaporated the solvent in ink to result in conductive traces on the LIG surface. The obtained Ag/LIG electrode immersed in the FeCl3 solution for 5 min was rinsed in the DI water and dried at room temperature. Coating three drops of Nafion on the electrode completed the fabrication of Nafion/AgCl/Ag/LIG RE. The fabrication of the WE relied on the electroless plating of Ni with the commercial Ni precursor solution (RTM Kit, Transene Company) which typically involves four steps: etching, surface sensitizing, fixing, and deposition (Loto 2016; Xu et al. 2018). However, the porous structure of LIG ensures surface roughness, which removes the need for the etching process. After surface sensitizing with palladium chloride solutions and fixing with sodium hypophosphite solutions for 4 min, the LIG sample is immersed in nickel sulfate solutions at 75° C. for the Ni electroless plating process. Due to the roughness difference between the LIG and PI, the Ni electroless plating prefers to occur on the LIG surface, which also eliminates the need for masks to define the electroplating region. A solution containing sodium gold sulfite (˜10% wt), ethylenediamine (˜5% wt), and potassium fluoride (5% wt) is used for the electroless Au plating at 80° C. The thickness of the Ni layer was controlled by the plating time. After rinsing with the DI water and drying at room temperature, a thin Au layer was electroless plated on the Ni/LIG electrode with the commercial Au precursor solution (Immersion Gold CF, Transene Company). Each electrode was fabricated and characterized separately before sensor integration. The Raman spectrum, SEM images, XRD, XPS, FTIR, and TEM-EDS were obtained by Horiba (HORIBA, Ltd.), NanoSEM 630 (FEI Company), Malvern Panalytical Empyrean III (Malvern Instruments, Ltd.), VersaProbe II (ULVAC-PHI, Inc.), Vertex 80 (Bruker Corp), and Talos TEM (The Thermo Scientific™), respectively.

Fabrication of the Compact Glucose Sensors with an Encapsulated Reaction Cavity

After separate characterization and validation of the WE and RE, a compact glucose sensor with a concentric three-electrode configuration was fabricated on a PDMS substrate (Sylgard 184, a mixing ratio of 1:10). Next, a porous encapsulated reaction cavity was prepared with PDMS. Briefly, the fabrication process started with the cutting of a cavity in a PDMS slab (i.e., the reaction member) by a CO₂ laser. After applying a layer of salt crystals at the bottom of the cavity to half of the cavity thickness, the Ecoflex diluted with heptane (w/w=1:1) was poured into the cavity to just immerse the salt layer. After the Ecoflex was cured, the salt crystals in the cavity were dissolved by the DI water followed by drying, leaving a porous polymer layer at the bottom. After flipping over the reaction cavity with the porous polymer layer on top, it was then sealed with the compact glucose sensor by a silicone glue. Injection of the NaOH solution of 0.05 M into the reaction cavity with a syringe completed the fabrication of the encapsulated glucose sensor.

### Electrochemical Characterization of the Non-Enzymatic Sensor

After connecting the three electrodes to an electrochemical workstation (PGSTAT302N, Metrohm Autolab) with copper foils and silver pastes, the electrochemical measurements were carried out in the 0.05 M NaOH. Magnetic bars were used during the addition of glucose solutions to ensure well mixing. Attaching the encapsulated glucose sensor over a plastic tube with a radius of 5 mm measured its sensing performance upon bending. After obtaining the calibration curve in the glucose concentration versus current density, the encapsulated glucose sensor was first applied to measure the glucose concentration in the cell culture media. Next, the glucose concentration of glucose solutions or human sweat was measured by dropping them onto the porous encapsulation. After attaching the encapsulated glucose sensor to the arm of healthy human subjects, the glucose concentration in sweat was measured to demonstrate its on-body performance. The glucose measurements from the encapsulated glucose sensor were compared to those from the commercial glucose sensors (Accu Chek Performa) where appropriate.

Integration of the Non-Enzymatic Sensor with Microfluidic Channels

The sensor was composed of three layers: a top layer, an intermediate layer (comprising a microfluidic channel and a glucose sensing layer), and a bottom layer. The microfluidic channel was fabricated by pouring Polydimethylsiloxane (PDMS; Sylgard 184, Dow Corning, US) with a mixing ratio of 1:10 into an acrylic plate mode fabricated by laser scribing (Universal Laser System Inc.). After cured, it was treated with 1-minute air plasma and 3 minutes 10% (w/v) PVP treatment to improve the hydrophilicity. Openings for the sweat inlet on the microfluidic channel and the glucose sensing layer were manually punched. Afterward, a top layer from the spinning coating on a silicon wafer was attached to the microfluidic channel with a thin PDMS adhesive. The glucose sensor on PI was attached to the reaction cavity on the bottom surface of the channel layer with a PDMS adhesive. A bottom layer with a bottom inlet was laminated onto the sensor to achieve intimate contact to human tissues for efficient sweat sampling. After loading the electrolyte soaked in porous polymer layer in the reaction cavity, the opening on the top layer was sealed by adhesive tape.

### Results and Discussion

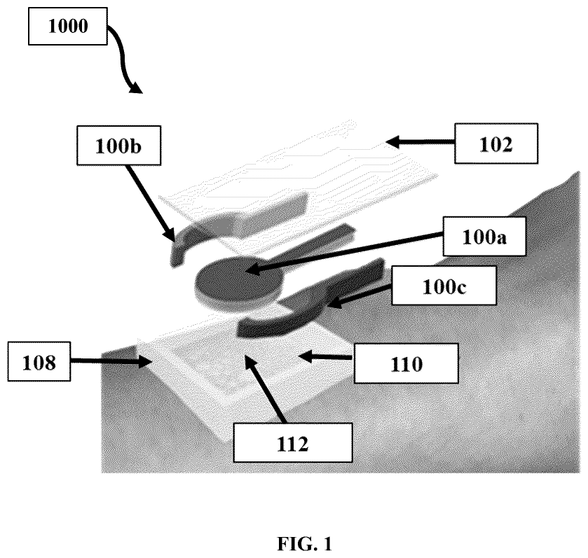

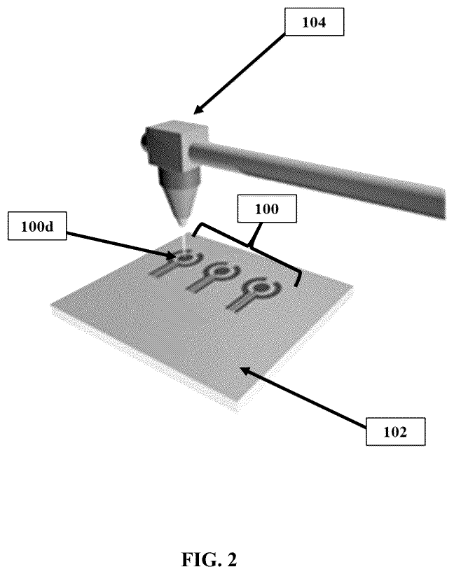

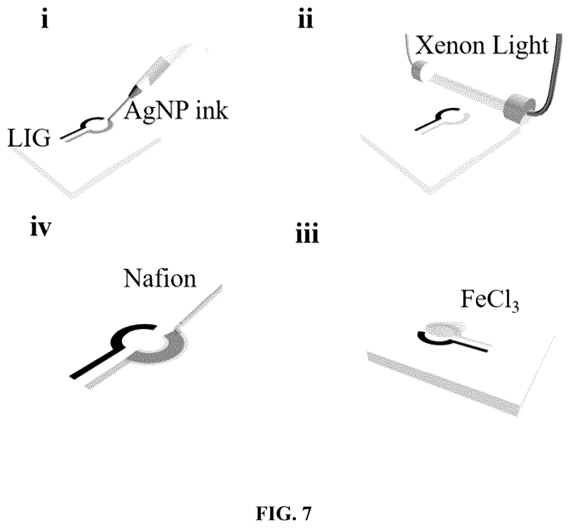

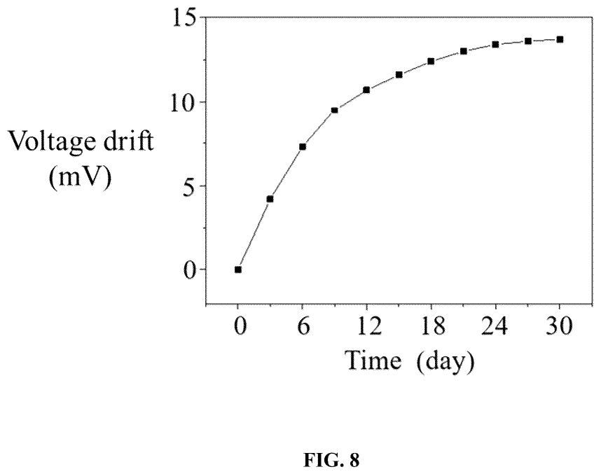

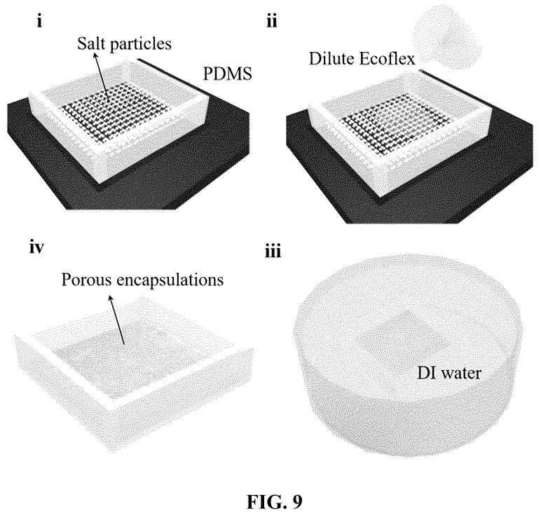

As discussed above, the Au/Ni/LIG WE is combined with the Nafion/AgCl/Ag/LIG RE and LIG CE electrodes in a three-electrode configuration to yield the non-enzymatic glucose sensor (FIG. 2). Briefly, the commercial thin PI films are first converted into LIG electrodes by a simple laser scribing process. After Ag coating on the LIG electrode surface and immersion in the FeCl₃ solution for chlorination, coating another layer of Nafion to get rid of the influence of chloride ions in the solution on the obtained electrode completes the fabrication of the Nafion/AgCl/Ag/LIG RE (FIG. 7). The potential measurement of the resulting RE only shows a ˜4 mV variation for 24 h even when immersed in PBS solutions with 0.1 M NaCl. A small variation of ˜13 mV was observed for 30 days' measurement (FIG. 8), indicating the long-term stability for electrochemical applications. The Au/Ni/LIG WE is obtained by electroless plating of Ni and Au on LIG electrodes in commercial pre-cursor solutions. After integrating the Au/Ni/LIG WE with the RE and CE, the compact glucose sensor can be encapsulated by a porous encapsulated reaction cavity (FIG. 9) for on-body wearable applications (FIG. 1).

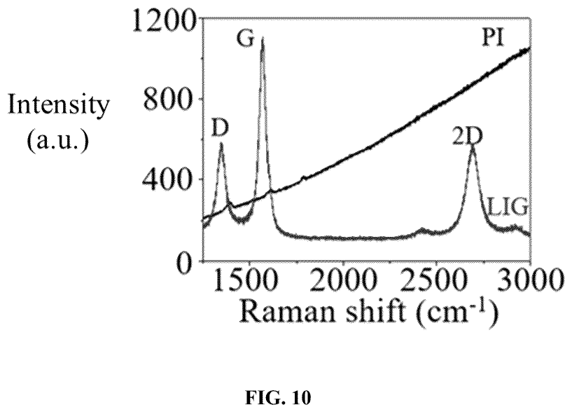

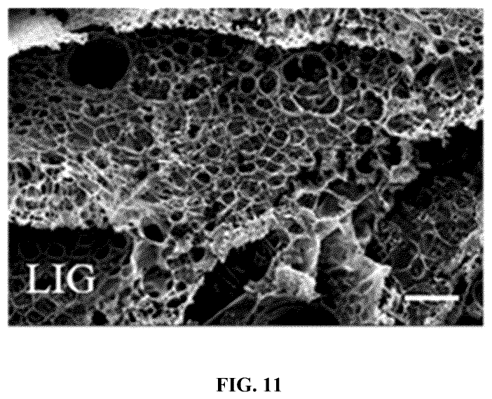

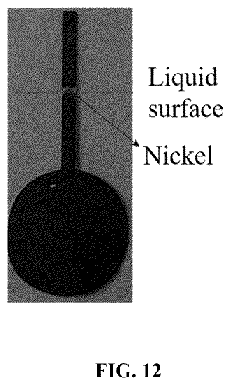

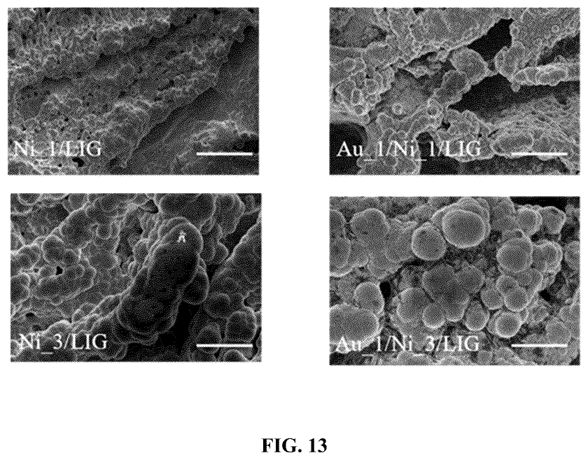

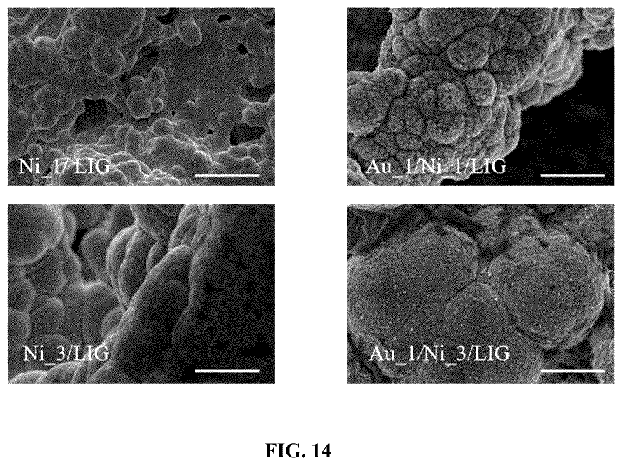

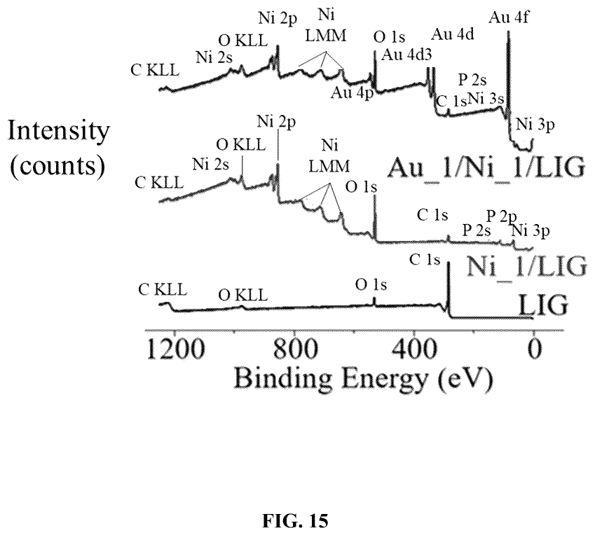

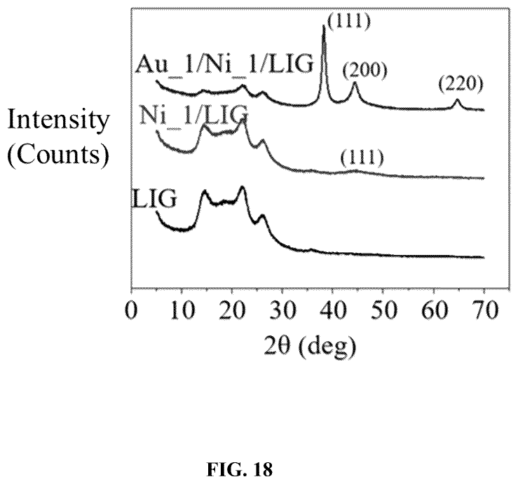

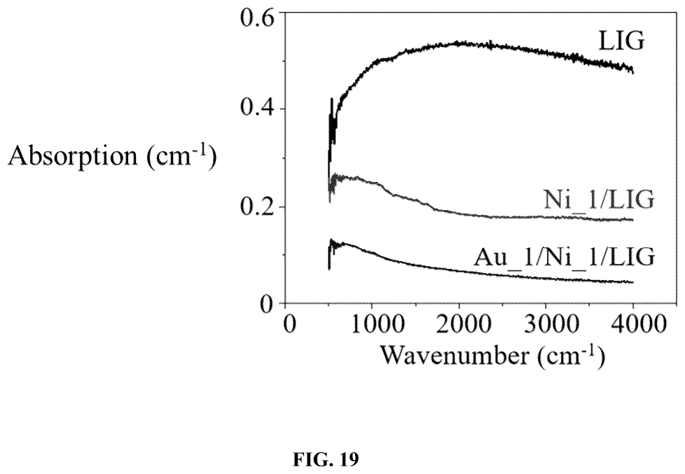

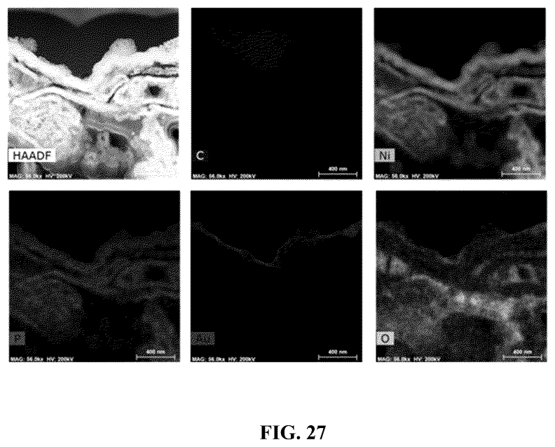

Before the electrochemical characterization of the integrated glucose sensor, the performance of individual electrodes is demonstrated and tested first. The Raman spectrum with three characteristic peaks (D peak at 1350 cm⁻¹, G peak at 1580 cm⁻¹, and 2D peak at 2700 cm⁻¹) and the ratio of the 2D to G peak of ca. 0.48 confirms the existence of multi-layered graphene with some defects (FIG. 10). These defects possibly originate from oxidation during the laser scribing without an inert gas environment. The small intensity ratio (˜0.5, FIG. 10) of the D to G peak implies a relatively high quality of the porous 3D graphene foam with a pore size of around 1-5 μm (FIG. 11). In contrast, these characteristics are not observed in the Raman spectrum of the PI, indicating the successful conversion of the PI to LIG by laser scribing. Although the obtained LIG with good conductivity (sheet resistance of ˜25Ω/□ or conductivity of ˜2000 S/m) is sufficient for capacitors and gas sensing platforms, it is still challenging to electroplate a uniform layer of sensing materials on the porous LIG electrodes. In fact, electroplating of nickel preferentially takes place at the LIG/solution interface, leaving the solution immersed LIG unplated (FIG. 12). Also, LIG flakes near the clamp often fall off during electroplating to result in failed plating processes. To address these challenges, we exploit the electroless plating to uniformly deposit a thin layer of Ni on the porous LIG electrode, where the thickness of the Ni layer is well controlled by the plating time. The color of LIG changes from black to grey with Ni coatings. With electroless plating of 1 min, the LIG foam is uniformly covered by a Ni layer without altering or blocking the porous structure (FIGS. 13 and 14). The structure with well-preserved porosity ensures a high specific surface area for fast diffusion of sampling solutions to result in a fast response and high sensitivity. When the plating time is increased to 3 min (FIG. 13), the Ni layer gets thicker and almost blocks the porous structure (FIG. 14). Though it is difficult to directly measure the thickness of the Ni layer on the highly porous 3D LIG foam, the comparison in the pore size of the same LIG foam before and after electroless plating provides a good estimate of its thickness. As the pore size decreases from ˜1.5 to 0.3 μm after plating of 1 min, the thickness of the Ni layer is estimated to be ˜0.6 μm. The Ni layer nearly blocks the porous structure after plating for 3 min, indicating a thickness of ˜0.75 μm. When the electroplating time of Ni is reduced to 10 or 30 s, the porous structures of the 3D LIG foam are well maintained, as observed by SEM characterizations (FIG. 14). The influence of Ni thickness on the electrochemical sensing performance was investigated in the following part. Because some subjects have a nickel allergy, another Au layer is then electroless plated for 1 min on the Ni/LIG surface to minimize this side effect (FIG. 13). Because Au is also sensitive to glucose, the Au/Ni/LIG WE is expected to maintain high sensitivity as well. The electroless plating of Au is a very slow and self-limiting process. According to the datasheet, the thickness of Au after electroless plating of 1 min is ca. 40 nm. The successful deposition of Ni and Au on the LIG is confirmed by X-ray diffraction (XRD) (FIG. 18) and X-ray photoelectron spectroscopy (XPS) (FIG. 15). The XPS results also reveal the existence of O elements from the oxidation of LIG and the electroplating precursor. The scanning trans-mission electron microscopy (STEM) combined with energy-dispersive X-ray spectroscopy (EDS) further reveals a conformal Ni coating on the LIG, followed by a thin Au layer on the surface of the Ni layer (FIG. 27). The presence of sodium and phosphorus atoms results from the electrolyte residual and the Ni electroless plating according to 2Ni²⁺+8H₂PO₂⁻+2H₂O→2Ni₀ (s)+6H₂PO₃⁻+2H⁺+2 P (s)+3H₂ (g). It is worth noting that the impurities (with the atomic weight shown in Table S1) in the fabricated glucose sensor do not affect glucose sensing performance. Additionally, Fourier-transform infrared spectroscopy (FTIR) has been carried out to verify the non-existence of the organic functional group on Ni or Au after electroless plating (FIG. 19), indicating that the glucose sensing may solely arise from the metal element. The Ni 1/LIG WE with a different thickness of the Au layer is studied in the following experiment: Ni_1/LIG, Au_1/Ni_1/LIG, Au 2/Ni_1/LIG, and Au 3/Ni_1/LIG (the number after Au and Ni indicates the plating time in min). Different from the substrate-assisted electroless deposition based on a galvanic displacement reaction, where the adopted Zn substrate has a redox potential than Cu ions in the solution, the electroless plating of Ni and Au adopted in this work doesn't need another assistant electrode. Due to the contrast of roughness be-tween LIG and PI, Ni electroless plating prefers to take place on LIG, eliminating the need for masks to define the electroplating region.

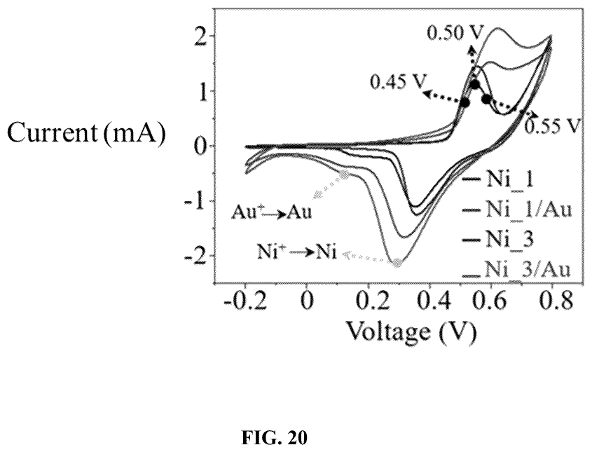

Without Au layers, the cyclic voltammetry (CV) curves of Ni/LIG electrodes (both 1 min and 3 min) reveal a single peak at ˜0.5 V (and −0.25 V) in the forward (and reverse) scan, which corresponds to the oxidation (and reduction) of Ni (FIG. 20). In a basic solution under a positive bias voltage, Ni(OH)₂ with Ni (II) arises from the oxidation of Ni anodes. The sensing mechanism of Ni to glucose relies on the following reaction:

Ni(OH)₂+OH⁻→NiO(OH)+H₂O+e⁻

NiO(OH)+Glucose→Ni(OH)₂+Glucolactone

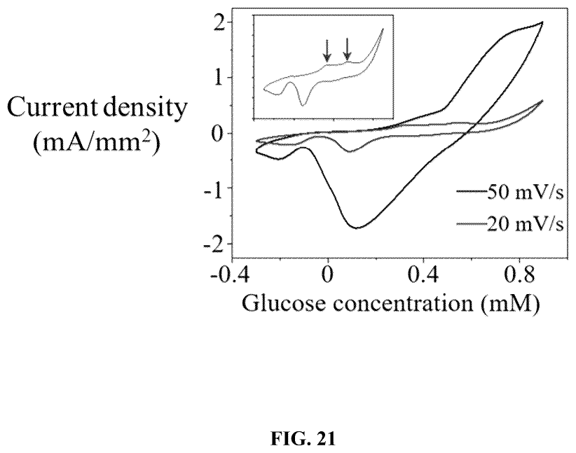

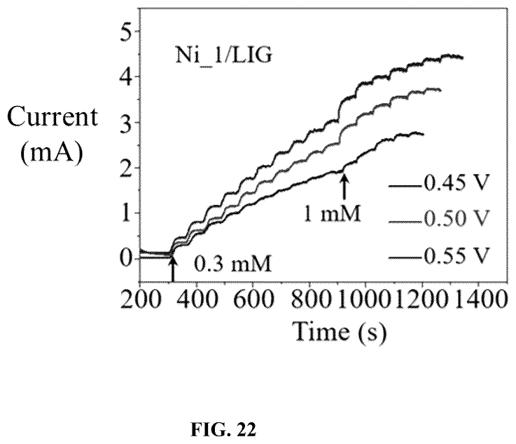

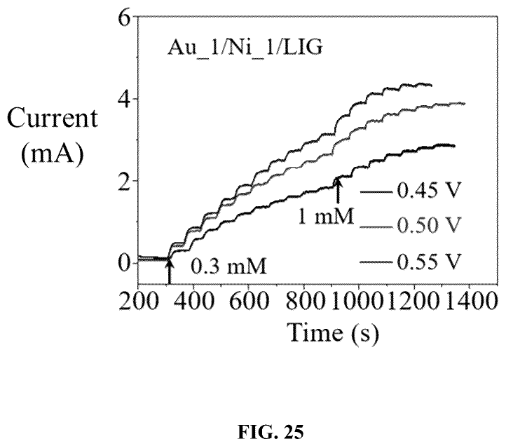

The introduction of the Au layer leads to another peak at ˜0.05 V in the reverse scan (light arrows in FIG. 20), which is consistent with previous reports and corresponding to the reduction of gold. The separate peak from Au indicates its active role as a reactive material in the electro-chemical reaction. The two clear peaks from the Ni and Au oxidations in the forward scan are not evident because they merge to form a single broad peak (see the curves at ˜0.5 V in FIG. 20). As the scan rate is reduced from 50 to 20 mV/s, two distinct oxidation peaks from the Ni and Au oxidations appear in the CV curve (FIG. 21). Because Au is less sensitive than Ni to glucose, the Au/Ni/LIG WE exhibits a reduced peak value in the CV curve. Next, the optimized bias voltage is determined before it is used to obtain the calibration curve of the current density versus glucose concentration by titration. By using three different bias voltages around the peak (i.e., 0.45, 0.50, and 0.55 V) for the Ni_1/LIG WE, the chronoamperometry (CA) measurement indicates the best sensitivity from a bias voltage of 0.55 V, followed by 0.5 V and then 0.45 V (FIG. 22). Because the Ni_1/LIG WE with the applied bias voltage of 0.5 V exhibits the highest signal-noise-ratio, the bias voltage of 0.5 V is chosen in the following CA measurement unless specified otherwise. The effect of the bias voltage on the Au_1/Ni_1/LIG electrode has also been investigated and the results similar to the Ni 1/LIG electrode have been observed (FIG. 25).

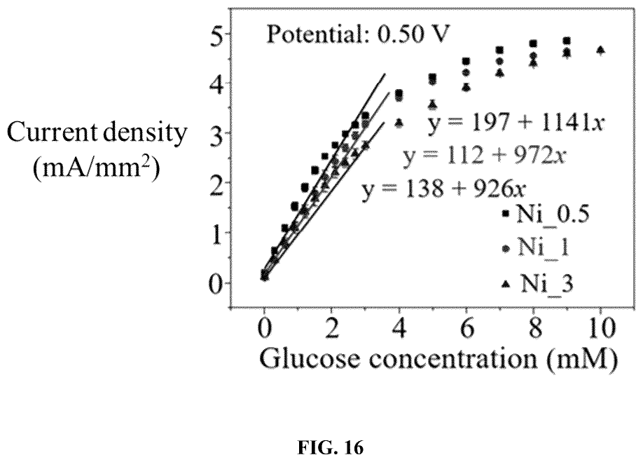

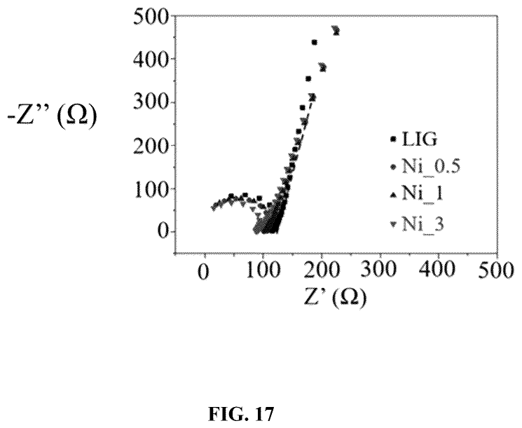

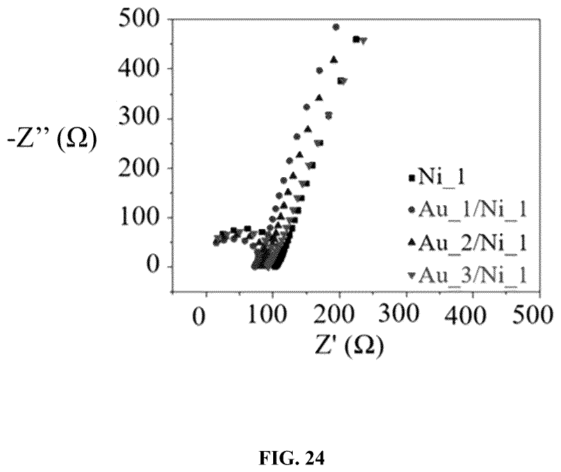

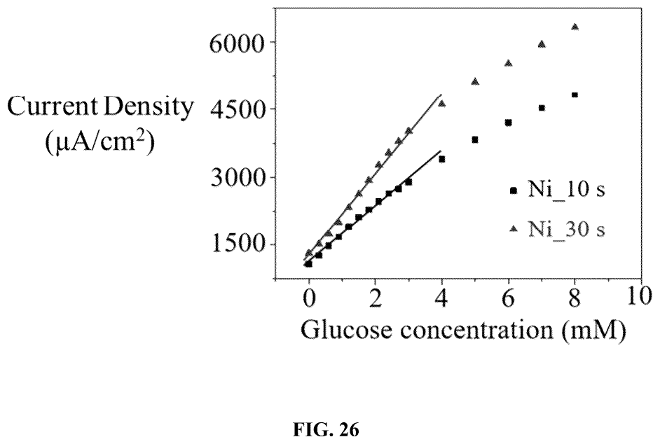

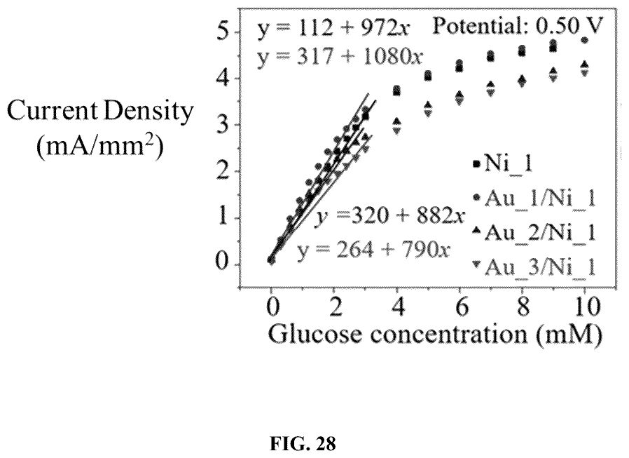

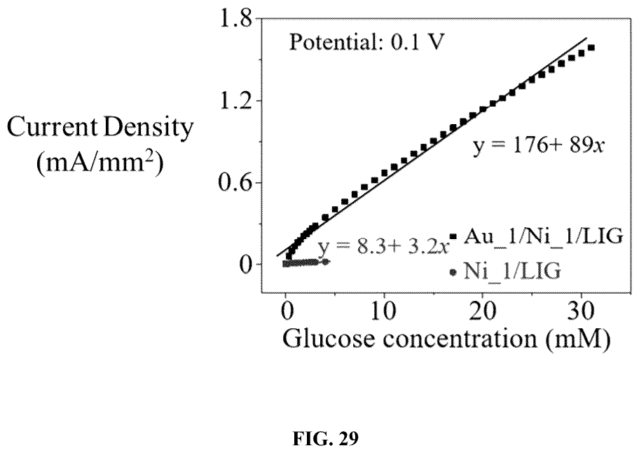

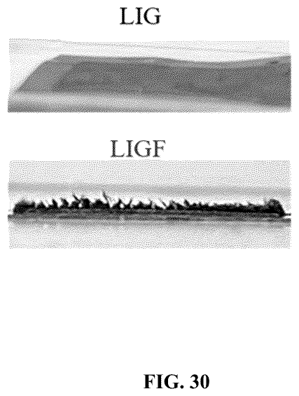

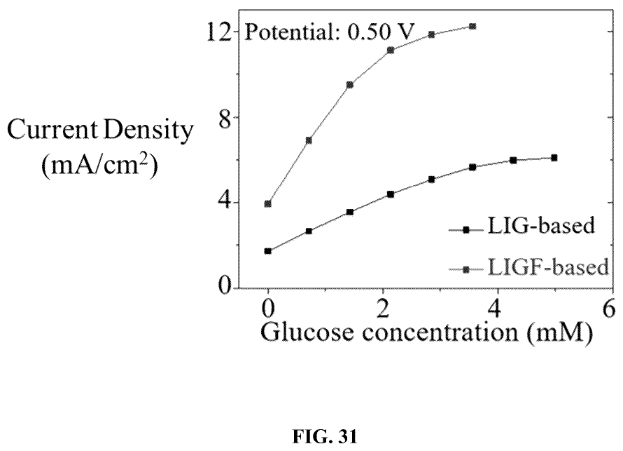

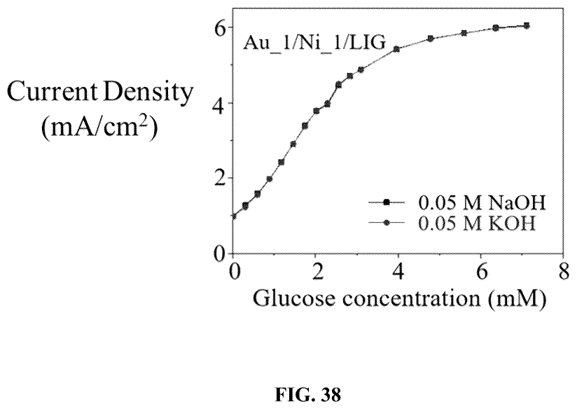

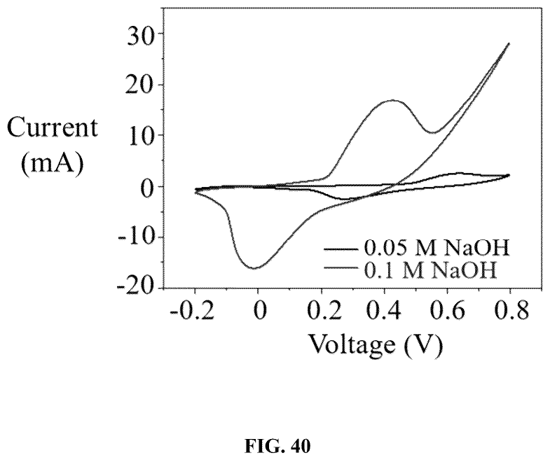

The current at the WE increases with the successive addition of glucose solutions until saturation and the response time for all four WEs is within 3 s. In the plot of the current density versus glucose concentration (FIG. 16), the slope of the linear range (with R²>0.99) gives the sensitivity, which gradually decreases with an increasing glucose con-centration. Because of the preserved porous LIG structure, the WE with a short deposition time of Ni shows higher sensitivity, e.g., 972 μA mM⁻¹ cm⁻² for the Ni_1/LIG WE and 926 μA mM⁻¹ cm⁻² for the Ni_3/LIG WE. Compared with other electrode materials such as Au or Pt, LIG is cost-effective and can be easily patterned without masks. The porous 3D LIG foam also provides the Ni/LIG electrode with improved sensitivity, as compared with the Ni electrode without 3D structures (241 μA mM⁻¹ cm⁻²). Reducing the deposition time from 1 min to 30 s further leads to an increased sensitivity of 1141 μA mM⁻¹ cm⁻². However, further reducing the deposition time to 10 s leads to a much-decreased sensitivity of 825 μA mM⁻¹ cm⁻² possibly due to the limited reactive materials (FIG. 26). These results indicate that there might exist an optimal deposition time between 10 and 60 s for the highest sensitivity. As the Ni plating time increases from 0.5 to 3 min, the circle radius in the electrochemical impedance spectroscopy (EIS) reduces, indicating a decreased charge-transfer resistance (FIG. 17). However, the diffusion rate indicated by the slope in the low-frequency range decreases with the Ni plating duration because a thick Ni layer tends to block the pore in LIG. As a result, the Ni_3/LIG WE has the lowest sensitivity due to the reduced specific surface area. Take the Ni_1/LIG WE as an example, the thickness of Au on the sensing performance is investigated (FIG. 28). 1-minute Au plating slightly improves the sensitivity from 972 μA mM⁻¹ cm⁻² in Ni_1/LIG to 1080 ρA mM⁻¹ cm⁻² in Au_1/Ni 1/LIG WE. With further increasing the Au plating time, the sensitivity decreases to 882 μA mM⁻¹ cm⁻² for the Au 2/Ni_1/LIG WE and 790 μA mM⁻¹ cm⁻² for the Au_3/Ni_1/LIG WE. The EIS measurement reveals that the Au coating helps to reduce the charge transfer resistance (FIG. 24). It is anticipated that the introduction of a thin Au coating increases the roughness of the Ni surface and further increases the specific surface area. While Au plating of 1 min improves the diffusion rate, increasing the plating time to 2 or 3 min exhibits an opposite effect due to the reduced reactivity of Au than Ni. However, increasing the Au layer thickness seems to block the porous structure, leading to a reduced specific surface area. The linear range of the Au_1/Ni_1/LIG glucose sensor is from 0 to ˜4 mM with a detection limit of 1.5 μM calculated from 3*SD/N, where SD and N is the standard deviation and slope of the linear fitting of the calibration curve, respectively. The demonstrated linear range is also able to cover the glucose concentration in sweat (0.01-2 mM), tears (0.5-5 mM), and saliva (0.55-1.77 mM). The Ni/LIG-based non-enzymatic glucose sensor exhibits a much wider linear range, compared with previously reported Ni nanoparticle-based ones (0.002-1.0 mM). It is also noted that the photo-reduction from the laser can be explored for laser-induced metals (e.g., nickel, gold, and copper) for non-enzymatic glucose sensing, which will be investigated in the future. The Au 1/Ni 1/LIG WE demonstrates a large linear range (0-30 mM) with a sensitivity of 89 μA mM⁻¹ cm⁻² under a bias voltage of 0.1 V due to the Au (0)/Au (I) redox (FIG. 29). The low bias voltage is beneficial for reduced power consumption and improved selectivity by reduced (or eliminated) potential oxidation of other species in the biofluids. The demonstrated sensitivity under a bias voltage of 0.5 V is ˜4 times of the non-enzymatic LIG glucose sensor (247.3 μA mM⁻¹ cm⁻²). After replacing the porous LIG foam with LIG fiber (LIGF, FIG. 30), the Au/Ni_1/LIGF glucose sensor with the same dimensions exhibits an even higher sensitivity of ˜3500 μA mM⁻¹ cm⁻² due to an increased specific surface area (FIG. 31). A comprehensive comparison of the sensing performance of the proposed and previously reported glucose sensors are made in Table 1. In summary, the non-enzymatic LIG glucose sensors demonstrated in this work compare favorably with the previous reports on Ni-based, Au-based, and bimetal-based glucose sensors (Table 1). In particular, the dual working modes (high sensitivity in a narrow linear range @ 0.1 V or low sensitivity in a wide linear range @ 0.5 V) can be of high interest for a diverse range of applications. Although the Au/Ni/LIG glucose sensor already showcases a favorable sensitivity and linear range in 0.05 M NaOH (pH=12.5), a higher reaction activity can be achieved in 0.1 M NaOH (pH=13) (FIG. 40), which can further improve the sensitivity and linear range. To demonstrate the applicability of the non-enzymatic glucose sensor in other basic solutions, 0.05 M KOH has also been explored as a representative example, which shows almost identical sensing performance (FIG. 38). Since the sensitivity of the Au/Ni_1/LIG glucose sensor in the 0.05 M NaOH solution is enough for measuring the glucose concentration in sweat, it is used in the following demonstrations for safer wearable applications unless otherwise specified. It should be noted that the other glucose sensors with higher sensitivity such as the one with Ni plating of 30 s or optimum time hold high potential to measure the breath glucose of ultralow concentration. Despite the highest sensitivity in the LIGF-based glucose sensor, the LIGFs with an average height of ˜2 mm make them prone to damage upon mechanical deformations. However, the encapsulation or integration strategies can be explored to use LIGF-based glucose sensors in future research.

TABLE 1

| | Sensitivity | | | | Real- |
|---|---|---|---|---|---|
| Electrode | (μA mM⁻¹ | LOD | Linear | Response | time |
| materials | cm⁻²) | (μM) | Range | time | sample |
| NiO | 30190 | 2 | 0.002-1.279 | N/A | no |
| microspheres | | | mM | | |
| Porous NiO | 5222 | 3.31 | 0.005-1.1 | N/A | no |
| | | | mM | | |
| Au foam | N/A | 0.14 | 0.0005- | N/A | human |
| | | | 12 mM | | serum |
| Au network | N/A | 0.2 | 1-500 μM | <2 s | no |
| Au/graphene | 97.8 | 0.062 | 0-10 mM | <3 s | no |
| Core-shell | 23.17 | 15.7 | 0.5-10 mM | <1 s | no |
| Au/Ni | | | | | |
| Au/Ni/Al | 1989 | 1 | 0.01-6.1 | <5 s | serum |
| | | | mM | | |
| Au/Ni | 30.58 | 5.84 | 0-30 mM | <1 s | blood |
| Au/Ni | 4035 | 0.2 | 0-3 mM | <3 s | no |
| dendrite | | | | | |
| Ni/Au | 3372 | 0.1 | 0.25-2 mM | <5 s | no |
| nanowire | | | | | |
| Au/Ni/LIG | 1200 | 1.5 | 0-4 mM @ | <1 s | sweat, |
| (LIGF) | (LIG) | | 0.5 V | | cell |
| | 3500 | | or | | culture |
| | (LIGF) | | 0-30 mM | | media |
| | | | @ 0.1 V | | |

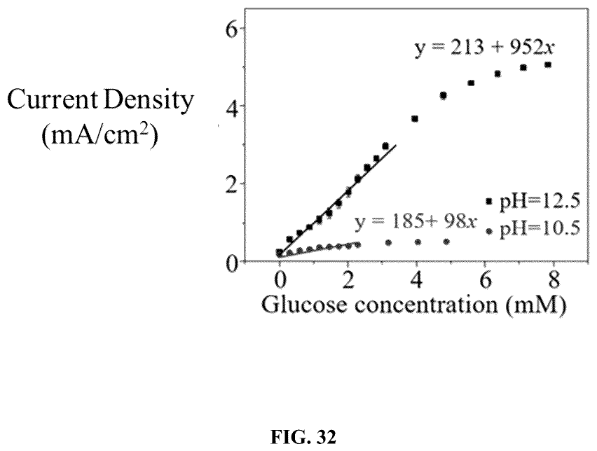

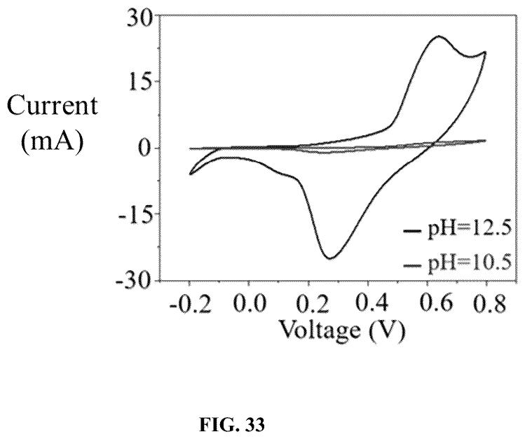

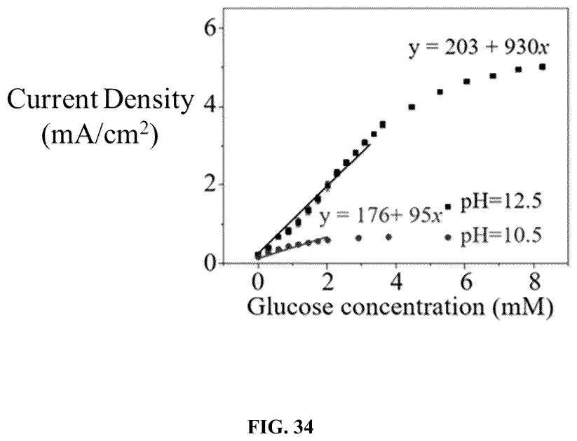

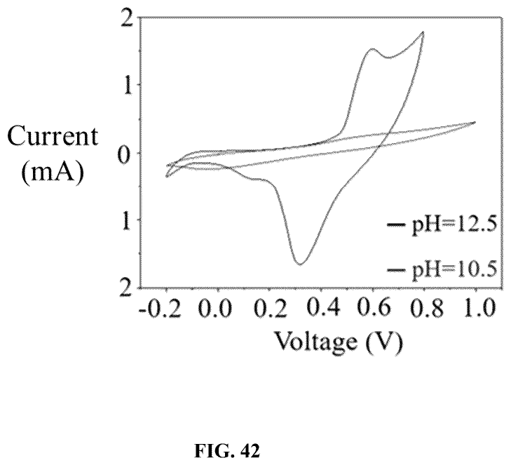

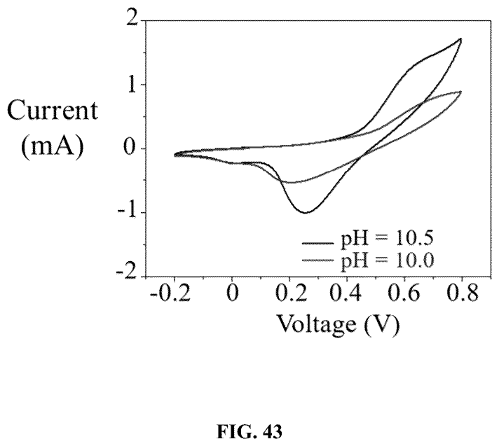

Because of the biofluids with varied pH levels, it is of high interest to investigate the sensing performance of non-enzymatic glucose sensors in solutions with different pH values. As the alkaline solution provides the hydroxide for the oxidation of glucose, the lower pH value from 12.5 to 10.5 in the basic solution results in a lower CV peak in the Au/Ni_1/LIG glucose sensor (FIG. 33). Further decreasing the pH from 10.5 to 10.0 leads to an almost vanishing CV peak (FIG. 43), implying a deactivated electrochemical reaction. These results imply that non-enzymatic glucose sensing is highly pH-dependent and it is essential to maintain a suitable pH environment for high-performance sensing. As a result of the lower CV peak, the sensitivity also significantly reduces from 952 to 98 μA mM⁻¹ cm⁻² (FIG. 32). The high sensitivity in less alkaline solutions is possibly attributed to the additional noble metal of Au. Though the requirement is significantly reduced in terms of the needed pH for the proposed sensor, the non-enzymatic glucose sensors still cannot be directly applied for measurements in human sweat that is near neutral or moderately acidic. Therefore, this example explores a reaction cavity with a porous Ecoflex containing 0.05 mM NaOH solutions to provide an alkaline environment for the non-enzymatic glucose sensor. When the porous encapsulated reaction cavity is filled with a solution with a pH of 12.5 or 10.5, the measured CV curves still exhibit the “dual-peak” in the reverse scan in the former and a smaller peak current in the latter (FIG. 42). The effectiveness of the porous encapsulated reaction cavity with the above two different solutions (i.e., pH of 12.5 or 10.5) in the encapsulated glucose sensor is demonstrated with the glucose measurements. The measured sensitivity of the encapsulated glucose sensor with the pH 12.5 (or 10.5) reaction cavity is 930 (or 95) μA·mM⁻¹ cm⁻² (FIG. 34), which is close to the value from the sensor in the corresponding open pH solution (FIG. 32). Therefore, the required alkaline environment in the sampling solution can be effectively removed when the encapsulated non-enzymatic glucose sensors are exploited. Furthermore, the sweat of 1000 mL with a pH of 6 will need to be added into the porous encapsulated reaction cavity with a pH of 12.5 to reduce its pH down to 10, which implies a longer operation time of the encapsulated glucose sensor when used for on-body wearable measurements.

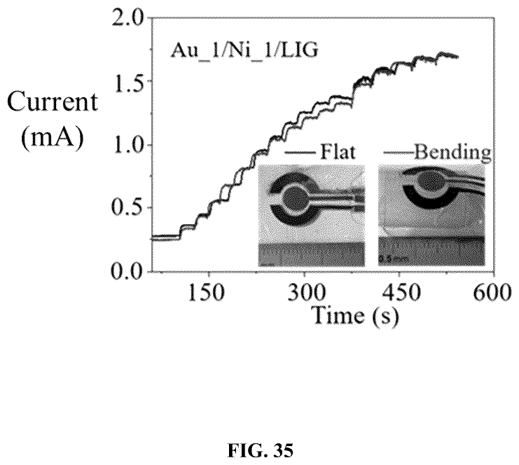

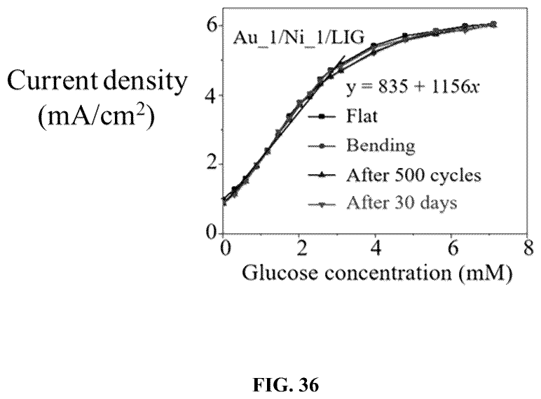

Because of the intrinsically flexible property of the LIG WE/RE/CE integrated on a soft PDMS substrate, the resulting flexible, the compact glucose sensor is able to accommodate various bending deformations without performance change in glucose sensing. Attaching the flexible glucose sensor to a cylinder tube with a radius of 5 mm indeed show-cases a small to negligible change in the response time, sensitivity, and measurement range (FIG. 35). Compare to the flat Au_1/Ni_1/LIG sensor with a sensitivity of 1156 μA mM⁻¹ cm 2, the measured sensitivity of 1123 μA mM⁻¹ cm⁻² only shows a 2% variation (FIG. 36). The almost unchanged sensitivity is also observed after the cyclic bending of 500 cycles to indicate cyclic stability of the sensor (FIG. 36). After the sensor immersion in PBS solutions with 0.1 M NaCl for 30 days, the Au 1/Ni 1/LIG WE exhibits almost unchanged sensitivity (FIG. 36).

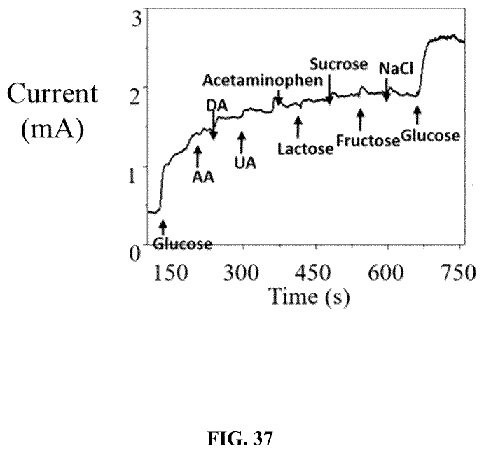

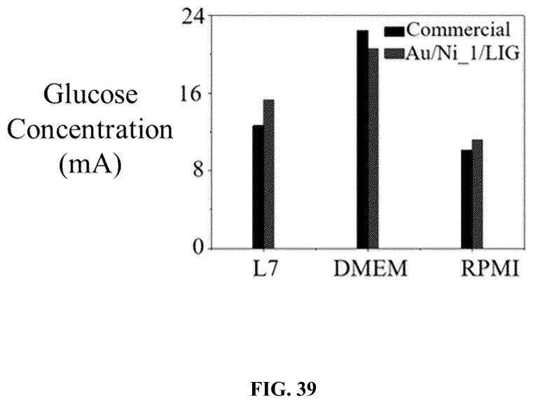

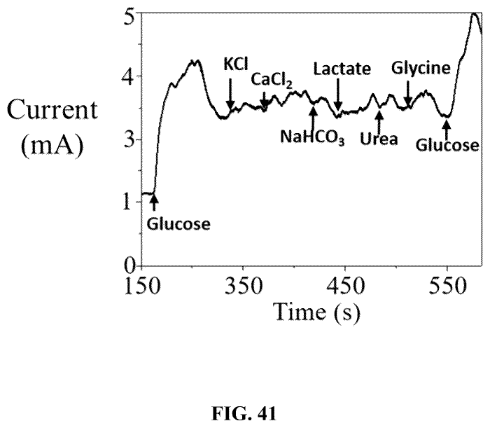

Although the selectivity of the non-enzymatic glucose sensors is often less than their enzymatic counterparts, our Au/Ni/LIG glucose sensor still highlights excellent selectivity against various interferences. Compared to the significant response to glucose (0.1 mM), little or almost no response is observed to these interfering substances, including 0.02 mM ascorbic acid (AA), dopamine hydrochloride (DA), uric acid (UA), acetaminophen, lactose, sucrose, fructose, lactate, urea, 1.5 mM glycine, and 0.2 mM NaCl, KCl, CaCl₂), and NaHCO₃ solutions (FIGS. 37 and 41). The good specificity of the demonstrated glucose sensor in this work allows it to accurately measure the glucose concentration of the sample with complex interfering substances. As a demonstration, the non-enzymatic glucose sensor accurately measures the glucose concentration in various cell culture media (FIG. 39). Despite the complex composites (e.g., salts, amino acids, glucose, and cell-secreted factors) in the media for stem cell culture, the glucose concentrations of the off-the-shelf cell culture media (DMEM and RPMI) measured by our glucose sensor agrees with those from the commercial glucose sensors very well. The measured glucose concentration of 21 mM (or 15 mM) in DMEM (or RPMI) with the flexible glucose sensor is reasonably close to that measured by the commercial one of 23 mM (or 13 mM). After the medium of L7 is used for cell culture for 24 h, the glucose concentration of 15 mM measured with our flexible glucose sensor still agrees with the value of 13 mM from the commercial one. Although the accuracy of the flexible glucose sensor can be further enhanced with a more accurate titration process, the accuracy in measuring glucose concentration is reasonably well in this proof-of-concept demonstration.

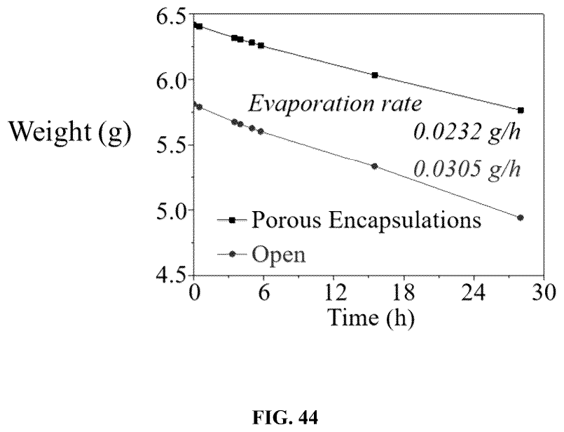

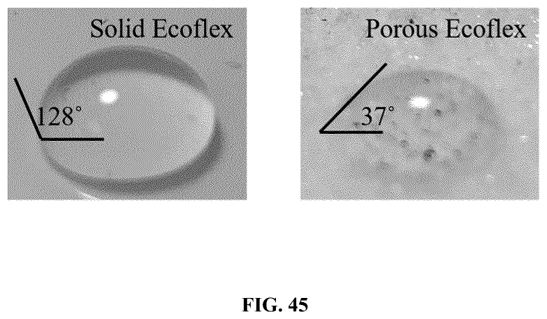

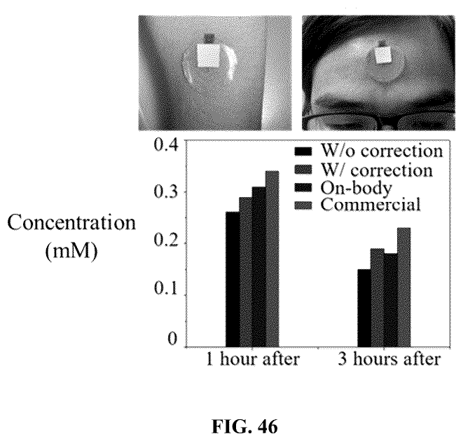

In the sensor, the porous encapsulated reaction cavity not only retains NaOH solutions to provide a relatively stable pH environment but also reduces evaporation while allowing sweat diffusion. The volume of NaOH electrolytes was carefully calibrated by micropipettes and kept fixed before glucose measurements. Compared to the design with an open cavity, the porous cavity reduces the evaporation rate by ˜24% (FIG. 44). The reduced evaporation can allow the glucose sensor to achieve long durability, which is especially important for wearable applications. The porous polymer layer also exhibits much-improved hydrophilicity, manifested by a much-reduced contact angle of 37° compared to the solid slab of 128. (FIG. 45). As a result, the porous polymer layer facilitates the diffusion of the sampling solution with spontaneous wicking. Further-more, the porous encapsulation is able to retain the NaOH solution within the cavity between it and the compact sensor, even when the encapsulated glucose sensor is flipped over or deformed from bending. Because of the well-maintained alkaline environment, the encapsulated glucose sensor can still provide accurate and reliable measurements of the glucose concentration in the prepared solutions (FIG. 46). Compared to the measurement in an electro-chemical cell, a longer response time is observed because of the diffusion barrier. Perforation of the porous polymer layer with small patterned holes by laser cutting reduces the response time from ˜6 min to 4 min while leaving the capability to retain the NaOH solution unaltered.

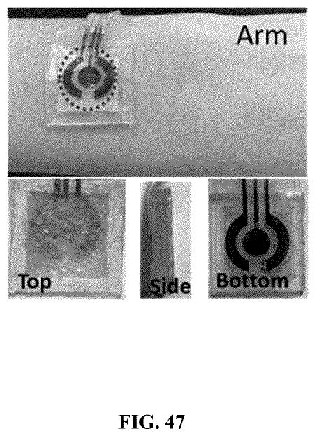

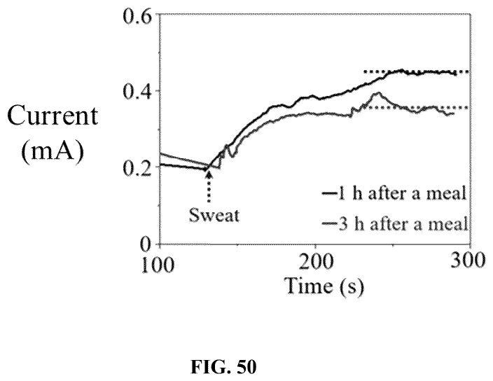

Next, the on-body performance is demonstrated by attaching the encapsulated glucose sensor to the arm of a healthy human subject after a workout (rowing machine) with an additional adhesive layer of Silbione (FIG. 47). The glucose analysis in sweat is first performed either 1 or 3 h after lunch from one healthy human subject (FIG. 50). The glucose concentration is obtained by adding 0.05 mL sweat samples to the reactive cavity for measuring the current response. The measurement indicates that the glucose concentration in sweat decreases from 0.26 mM to 0.15 mM. Even though the sweat volume is much smaller than that of the NaOH solution in the reaction cavity, the long-term use with excessive sweat may change the pH value of the NaOH solution. Therefore, it is crucial to monitor the pH value in the reaction cavity or in the sweat to help inform the pH change in the reaction cavity.

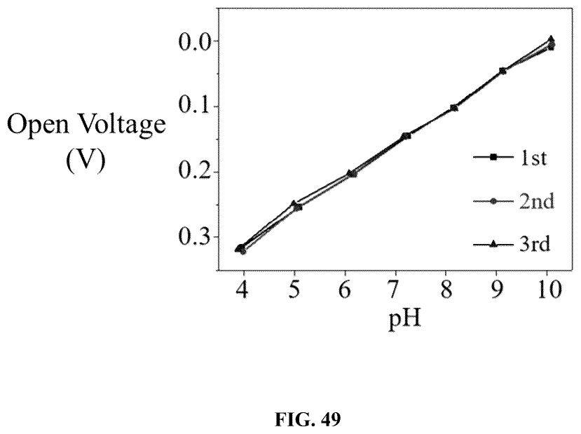

Demonstration of this idea includes an additional WE prepared by depositing polyaniline (PANI) on LIG. The pH value is determined from the potential difference between the PANI/LIG WE and the RE (FIG. 49). With the measured pH value in real-time, the glucose concentration obtained from the calibration curve at the given pH value provides a more accurate result. In fact, the sweat glucose concentration of 0.29 mM (0.19 mM) calculated at the measured pH level is indeed higher than that of 0.26 mM (0.15 mM) calculated at the initial pH level, which agrees better with the measurements from the commercial glucose sensors (FIG. 46).

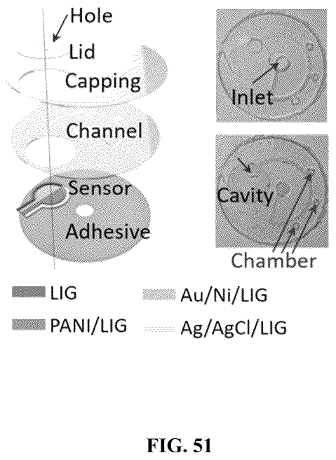

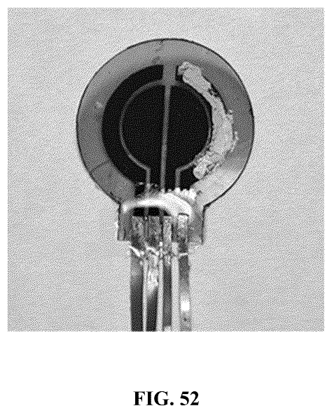

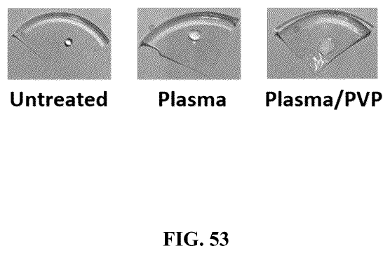

The wearable non-enzymatic glucose sensor can also be integrated with a soft, skin-interfaced microfluidic component for real-time sweat sampling and glucose analysis (FIG. 51). The electrical connection from the electrodes of the glucose sensor to the electrochemical station is made with thin copper wires and silver paste, which is further encapsulated by a thin layer of PDMS to improve mechanical robustness (FIG. 52). In the soft microfluidic component, the intermediate layer (comprising a microfluidic channel layer and a glucose sensing layer) is sandwiched between a top capping layer and a bottom layer that can pliably attach to the skin. The porous polymer layer containing the NaOH solution is replaceable in the reaction cavity to provide the basic environment for glucose sensing. Because of the improved hydrophilicity in the channel layer after the air plasma and PVP treatment to create C═O and —OH groups (FIG. 53), the sweat can be spontaneously wicked into the microfluidic channel for efficient sweat sampling. The inlet with a diameter of 3 mm covers ˜9 sweat glands, which also leads to rapid sweat collection from the human arm or forehead. The average sweat flow rate of 0.4 μL/min per sweat gland after a 7-min workout with a rowing machine is calculated from the ratio of the channel volume (0.576 μL) between two chamber markers (arc length of 4.8 mm) to the corresponding filling time (9.6 s). The sweat volume collected into the reaction cavity is the product of the sweat flow rate and the time interval. The glucose con-centration in sweat measured by the integrated microfluidic glucose sensor shows a consistent decrease from 0.31 to 0.20 mM, which agrees with the measurements of 0.34 and 0.23 mM from the commercial glucose sensor (FIG. 46). The use of the replaceable porous polymer layer in the reaction cavity allows the replacement of the NaOH solution and cleaning of the microfluidic channel with the distilled water, which regenerates the integrated device for reuse. The regenerated device provides the measured glucose concentrations of 0.30 mM and 0.21 mM, which are consistent with measurements from the freshly prepared de-vice. By exploiting multiple sensing chambers with hydrophobic valves, the sweat can be sequentially collected into each sensing chamber along the microfluidic channel at different times for chrono-sampling and glucose sensing.

The paired t-test was performed to evaluate the measured data for three cell media and two sweat samples.

TABLE 2

| | Au_1/Ni/LIG | Commercial |
|---|---|---|
| Samples | Mean (SD) (mM) | |
| L7 | 15.0 (±1.1) | 13.2 (±1.2) |
| DMEM | 21.1 (±1.5) | 22.8 (±1.7) |
| RPMI | 9.9 (±0.71) | 8.5 (±0.8) |
| Sweat 1 | 0.29 (±0.02) | 0.34 (±0.04) |
| Sweat 2 | 0.19 (±0.01) | 0.23 (±0.02) |

Each sample was measured three times. The t-value is calculated to be 0.58 and 1.35 for the cell media and sweat sample, which is much smaller than the critical value (1.76 or 2.02) at the 95% confidence level (n=9 or 6). The demonstrated non-enzymatic glucose sensor could also be integrated with wireless measurement units to provide real-time monitoring capabilities.

### CONCLUSION

This example demonstrates the use of 3D porous LIG foams or fibers on a flexible, thin-film substrate and electroless plating of Ni and Au for high-performance non-enzymatic glucose sensors. With well-maintained porosity in the 3D LIG foam scaffold, the resulting glucose sensor exhibits high sensitivity. The Au/Ni co-plating imparts the glucose sensor dual working modes: a higher sensitivity in a smaller linear range or a relatively lower sensitivity in a larger linear range. The sensitivity is further improved by 3.7 times when the LIG foams are replaced by fibers in the working electrode. Before the use of the porous encapsulated reaction cavity, the non-enzymatic glucose sensor still demonstrates a higher sensitivity in the relatively mild basic solution (pH=10) than those in the strong basic solutions in the literature, attributing to the Au layer. The demonstrated sensitivity and linear range compare favorably with those in the literature reports. Moreover, the encapsulated glucose sensor with a reaction cavity to provide a mildly alkaline environment further removes the need for an alkaline sampling solution. The exceptional sensitivity and selectivity of the LIG-based non-enzymatic glucose sensor ensure precise measurement of the glucose concentration in the complex sampling solutions (e.g., stem cell culture media and human sweat). In addition to robustly retain the mild NaOH solution inside the reaction cavity even upon flipping over or severe deformations, the porous Ecoflex encapsulation with improved hydrophilic properties also facilitates the diffusion of the sampling solutions and reduces the evaporation for faster and more accurate measurements. By integrating a microfluidic channel for sweat sampling, the non-enzymatic glucose sensor can be used in a wearable form, providing an alternative way for sweat glucose measurement. The integrated system including of the encapsulated non-enzymatic glucose sensor with excellent on-body performance and other wearable sensors, as well as wireless data collection and communication units, opens up additional opportunities in future wearable devices for biomedicine.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the device and methods of making and using the same have been discussed and illustrated herein, it is to be distinctly understood that the invention is not limited thereto but may be otherwise variously embodied and practiced within the scope of the following claims.

What is claimed is:

1. A non-enzymatic glucose sensor comprising:
one or more electrodes;
a microfluidic channel; and
at least one inlet, wherein the at least one inlet is configured to deliver a fluid to the microfluidic channel and wherein the microfluidic channel is configured to transport the fluid to the one or more electrodes,
wherein at least one of the one or more electrodes is a laser-induced graphene electrode, wherein the laser-induced graphene electrode comprises a uniform coating of a metal.

2. The non-enzymatic glucose sensor of claim 1, wherein the laser-induced graphene electrode further comprises a second uniform coating of a second metal, and wherein the second uniform coating is coated on the uniform coating of the metal.

3. The non-enzymatic glucose sensor of claim 2, wherein the metal comprises nickel the second metal comprises gold.

4. The non-enzymatic glucose sensor of claim 2, further comprising a reaction member comprising a reaction cavity positioned within the reaction member, wherein the reaction member is attached to the one or more electrodes such that reaction cavity is encapsulated.

5. The non-enzymatic glucose sensor of claim 4, wherein the reaction member further comprises an electrolyte solution positioned within the reaction cavity.

6. The non-enzymatic glucose sensor of claim 5, wherein the electrolyte solution has a pH between 8 and 12.5.

7. The non-enzymatic glucose sensor of claim 5, wherein the electrolyte solution comprises NaOH.

8. The non-enzymatic glucose sensor of claim 5, wherein the reaction member further comprises a porous polymer layer positioned within the reaction cavity, and wherein the electrolyte solution is positioned within the porous polymer layer.

9. A non-enzymatic glucose sensor comprising:
a top layer;
a bottom layer; and
an intermediate layer comprising:
one or more electrodes;
a microfluidic channel; and
at least one intermediate inlet, wherein the at least one intermediate inlet is configured to deliver a fluid to the microfluidic channel and wherein the microfluidic channel is configured to transport the fluid to the one or more electrodes,
wherein at least one of the one or more electrodes is a laser-induced graphene electrode, wherein the laser-induced graphene electrode comprises a uniform coating of a metal, and
wherein the intermediate layer is positioned between the top layer and the bottom layer and the bottom layer is configured to be in contact with a user.

10. The non-enzymatic glucose sensor of claim 9, wherein the laser-induced graphene electrode further comprises a second uniform coating of a second metal, and wherein the second uniform coating is coated on the uniform coating of the metal.

11. The non-enzymatic glucose sensor of claim 10, wherein the metal comprises nickel the second metal comprises gold.

12. The non-enzymatic glucose sensor of claim 9, wherein the bottom layer comprises at least one bottom inlet, wherein the at least one bottom inlet is configured to deliver a fluid to the at least one intermediate inlet.

13. The non-enzymatic glucose sensor of claim 9, wherein the bottom layer comprises an adhesive configured to attach to the user.

14. The non-enzymatic glucose sensor of claim 9, wherein the top layer is sealed to the bottom layer.

15. A method of forming a non-enzymatic glucose sensor, comprising:
providing a substrate;
providing a laser device;
laser-scribing the substrate using the laser device to form one or more laser-induced graphene electrodes;
depositing a uniform coating of a metal on at least one of the one or more laser-induced graphene electrodes; and
providing a microfluidic channel, wherein the microfluidic channel is configured to transport a fluid to the one or more electrodes.

16. The method of forming a non-enzymatic glucose sensor of claim 15, further comprising depositing a second uniform coating of a second metal on the uniform coating of the metal.

17. The method of forming a non-enzymatic glucose sensor of claim 16, wherein the metal comprises nickel and the second metal comprises gold.

18. The method of forming a non-enzymatic glucose sensor of claim 15, further comprising:
providing a reaction member comprising a reaction cavity positioned within the reaction member;
attaching the reaction member to the one or more electrodes such that reaction cavity is encapsulated;
providing an electrolyte solution positioned within the reaction cavity; and
providing a porous polymer layer positioned within the reaction cavity, wherein the electrolyte solution is positioned within the porous polymer layer.

19. The method of forming a non-enzymatic glucose sensor of claim 15, wherein the step of depositing a uniform coating of a metal on at least one of the one or more laser-induced graphene electrodes comprises electroless-plating of the metal on at least one of the one or more laser-induced graphene electrodes.

20. The method of forming a non-enzymatic glucose sensor of claim 16, wherein the step of depositing a second uniform coating of a second metal on the coating of the metal comprises electroless-plating of the second metal on the uniform coating of the metal.
